(19) United States
(12) Patent Application Publication
(10) Pub. No.: US 2024/0226130 A1
(43) Pub. Date: Jul. 11, 2024

(54) 4'-THIONUCLEOSIDE ANALOGUES AND THEIR PHARMACEUTICAL USE

(71) Applicant: Gilead Sciences, Inc., Foster City, CA (US)

(72) Inventors: Michael O. Clarke, Redwood City, CA (US); Bindu Goyal, Fremont, CA (US); Richard L. Mackman, Millbrae, CA (US); Devan Naduthambi, San Bruno, CA (US); Neil H. Squires, San Francisco, CA (US)

(21) Appl. No.: 18/479,576

(22) Filed: Oct. 2, 2023

Related U.S. Application Data
(60) Provisional application No. 63/413,192, filed on Oct. 4, 2022.

(51) Int. Cl. A61K 31/7076; A61K 31/7068; A61K 31/708; A61K 45/06; A61P 31/18; C07H 19/06; C07H 19/10; C07H 19/16; C07H 19/207

(52) U.S. Cl. CPC A61K 31/7076; A61K 31/7068; A61K 31/708; A61K 45/06; A61P 31/18; C07H 19/06; C07H 19/10; C07H 19/16; C07H 19/207

(57) ABSTRACT

Compounds, compositions, and methods useful for treating a viral infection, such as human immunodeficiency virus (HIV) infection, are disclosed. In particular, 4′-thionucleoside analogues and methods for their preparation and use as therapeutic or prophylactic agents are disclosed.

### CROSS-REFERENCE TO RELATED APPLICATIONS

The present application claims the priority benefit of U.S. Provisional Application No. 63/413,192, filed Oct. 4, 2022, which is hereby incorporated by reference in its entirety.

### FIELD

Compounds, compositions, and methods useful for treating a viral infection, such as human immunodeficiency virus (HIV) infection, are disclosed.

### BACKGROUND

Human immunodeficiency virus infection and related diseases are a major public health problem worldwide. Human immunodeficiency virus encodes three enzymes that are required for viral replication: reverse transcriptase, protease, and integrase. Drugs targeting reverse transcriptase are in wide use and have shown effectiveness, particularly when employed in combination with, for example, protease inhibitors and integrase inhibitors. No HIV cure is known, and accordingly, those affected by HIV can require life-long treatments. Improved treatments for HIV and other viral infections are desirable.

### SUMMARY

The present disclosure is directed to novel nucleoside inhibitors of HIV reverse transcriptase and pharmaceutically acceptable salts thereof. In some embodiments, the compounds may be used to treat HIV infections, to inhibit the activity of HIV reverse transcriptase, and/or to reduce HIV replication. In some embodiments, the compounds may be used to prevent HIV infections. In some embodiments, compounds disclosed herein may be effective against a range of known drug-resistant HIV mutants. In some embodiments, compounds disclosed herein have properties that make it possible for them to be administered with less than daily frequency, for example, at weekly, monthly, or longer intervals.

In one embodiment, compounds having the following formula (I) or a pharmaceutically acceptable salt thereof are provided:

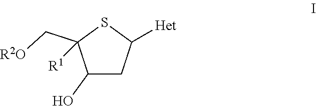

wherein

R¹ is C₂-C₈ alkynyl;

R² is selected from H,

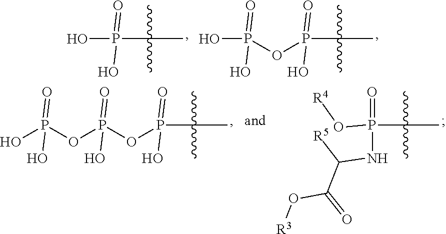

wherein:
R³ is C₁-C₈ alkyl;
R⁴ is selected from phenyl and naphthyl; and
R⁵ is H or C₁-C₆ alkyl; and

Het is

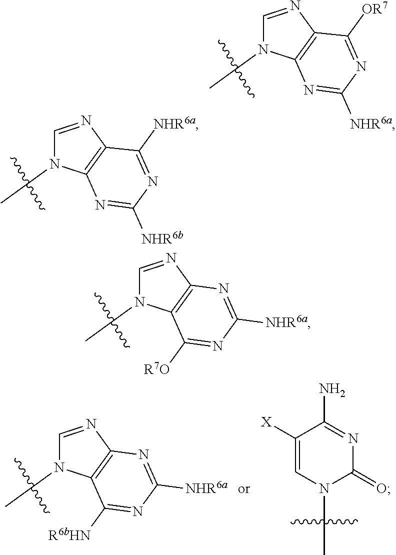

wherein:
X is halo;
R^{6a} is independently selected from H, C₁-C₆ alkyl, C₃-C₈ cycloalkyl, and C₁-C₆-alkylene-C₃-C₈ cycloalkyl;
R^{6b} is selected from H, C₁-C₆ alkyl, C₃-C₈ cycloalkyl, and C₁-C₆-alkylene-C₃-C₈ cycloalkyl; and
R⁷ is C₁-C₆ alkyl.

In another embodiment, a pharmaceutical composition is provided comprising a therapeutically effective amount of a compound of formula I, or a pharmaceutically acceptable salt thereof, and a pharmaceutically acceptable excipient.

In another embodiment, a kit or an article of manufacture is provided comprising a compound of formula I, or a pharmaceutically acceptable salt thereof, and instructions for use.

In another embodiment, a kit or an article of manufacture is provided comprising a pharmaceutical composition comprising a therapeutically effective amount of a compound of formula I, or a pharmaceutically acceptable salt thereof, and a pharmaceutically acceptable excipient, and instructions for use.

In another embodiment, a method of treating an HIV infection by administering to a subject in need thereof a therapeutically effective amount of a compound of formula I, or a pharmaceutically acceptable salt thereof, is provided.

In another embodiment, a method of treating an HIV infection, by administering to a subject in need thereof a pharmaceutical composition comprising a therapeutically effective amount of a compound of formula I, or a pharmaceutically acceptable salt thereof, and a pharmaceutically acceptable excipient, is provided.

In another embodiment, a method of preventing an HIV infection, by administering to a subject at risk thereof a therapeutically effective amount of a compound of formula I, or a pharmaceutically acceptable salt thereof, is provided.

In another embodiment, a method of preventing an HIV infection, by administering to a subject at risk thereof a pharmaceutical composition comprising a therapeutically effective amount of a compound of formula I, or a pharmaceutically acceptable salt thereof, and a pharmaceutically acceptable excipient, is provided.

In another embodiment, a compound of formula I, or a pharmaceutically acceptable salt thereof, for use in treating an HIV infection is provided.

In another embodiment, a pharmaceutical composition comprising a therapeutically effective amount of a compound of formula I or a pharmaceutically acceptable salt thereof, and a pharmaceutically acceptable excipient, for use in treating an HIV infection is provided.

In another embodiment, a method of using a compound of formula I in therapy is provided. In particular, a method of treating the proliferation of the HIV virus, treating AIDS, or delaying the onset of AIDS or ARC symptoms in a mammal (e.g., a human), comprising administering to the mammal a compound of formula I or a pharmaceutically acceptable salt thereof, and a pharmaceutically acceptable excipient, is provided.

In another embodiment, a composition comprising a compound of formula I or a pharmaceutically acceptable salt thereof, and a pharmaceutically acceptable excipient, for use in a method of treating the proliferation of the HIV virus, treating AIDS, or delaying the onset of AIDS or ARC symptoms in a mammal (e.g., a human) is provided.

In another embodiment, a kit or an article of manufacture comprising a composition effective to treat or prevent an HIV infection; and packaging material comprising a label which indicates that the composition can be used to treat or prevent infection by HIV, is provided. Exemplary compositions comprise a compound of formula I as disclosed herein, or a pharmaceutically acceptable salt thereof.

In another embodiment, a method of inhibiting the replication of HIV is provided. The method comprises exposing the virus to an effective amount of a compound of formula I or a salt thereof, under conditions where replication of HIV is inhibited.

In another embodiment, the use of a compound of formula I, or a pharmaceutically acceptable salt thereof to inhibit the activity of HIV reverse transcriptase is provided.

In another embodiment, the use of a compound of formula I, or a salt thereof, to inhibit the replication of HIV is provided. Other embodiments, may be set forth in the detailed description of the embodiments that follows, and in part may be apparent from the description, or may be learned by practice, of the claimed embodiments. These may be realized and attained by the processes and compositions particularly pointed out in the description and claims thereof. The foregoing Summary has been made with the understanding that it is to be considered as a brief and general synopsis of some of the embodiments disclosed herein, and is not intended to limit in any manner the scope, or range of equivalents, to which the appended claims are lawfully entitled.

### DETAILED DESCRIPTION

In the following description, certain specific details are set forth in order to provide a thorough understanding of various embodiments disclosed herein. However, one skilled in the art will understand that the embodiments disclosed herein may be practiced without these details. The description below of several embodiments is made with the understanding that the present disclosure is to be considered as an exemplification of the claimed subject matter and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### Definitions

Unless the context requires otherwise, throughout the present disclosure and claims, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment disclosed herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “about” or “approximately,” used in connection with a quantity is inclusive of the stated value and has the meaning dictated by the context (e.g., includes the degree of error associated with measurement of the particular quantity).

As used herein, the term “administering” or “administration” typically refers to the administration of a composition to a subject to achieve delivery of an agent that is, or is included, in a composition to a target site or a site to be treated. Those of ordinary skill in the art will be aware of a variety of routes that may, in appropriate circumstances, be used for administration to a subject, for example a human. For example, in some embodiments, administration may be parenteral. In some embodiments, administration may be by injection (e.g., intramuscular, intravenous, or subcutaneous injection). In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time). In some embodiments, administration may involve continuous dosing (e.g., perfusion) for at least a selected period of time.

The term “(C₁₋ₙ)alkyl” as used herein, wherein n is an integer, either alone or in combination with another radical, is intended to mean acyclic, straight or branched chain alkyl radicals containing from 1 to n carbon atoms. For example, “(C₁₋₆)alkyl” includes, but is not limited to, methyl, ethyl, propyl (n-propyl), butyl (n-butyl), 1-methylethyl (iso-propyl), 1-methylpropyl (sec-butyl), 2-methylpropyl (iso-butyl), 1,1-dimethylethyl (tert-butyl), pentyl and hexyl. The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group.

“Alkyl” is hydrocarbon containing normal, secondary or tertiary atoms. For example, an alkyl group can have 1 to 20 carbon atoms (i.e, (C₁-C₂₀)alkyl), 1 to 10 carbon atoms (i.e., (C₁-C₁₀)alkyl), 1 to 8 carbon atoms (i.e., (C₁-C₈)alkyl), 1 to 6 carbon atoms (i.e., (C₁-C₆ alkyl), or 1 to 4 carbon atoms (i.e., (C₁-C₄)alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 2-ethyl-1-butyl (—CH₂CH(CH₂CH₃)CH₂CH₃), and octyl (—(CH₂)₇CH₃). “Alkyl” also refers to a saturated, branched or straight chain hydrocarbon radical having two monovalent radical centers derived by the removal of two hydrogen atoms from the same or two different carbon atoms of a parent alkane. For example, an alkyl group can have 1 to 10 carbon atoms (i.e., (C₁-C₁₀)alkyl), or 1 to 6 carbon atoms (i.e., (C₁-C₆)alkyl) or 1-3 carbon atoms (i.e., (C₁-C₃)alkyl). Typical alkyl radicals include, but are not limited to, methylene (—CH₂—), 1,1-ethyl (—CH(CH₃)—), 1,2-ethyl (—CH₂CH₂—), 1,1-propyl (—CH(CH₂CH₃)—), 1,2-propyl (—CH₂CH(CH₃)—), 1,3-propyl (—CH₂CH₂CH₂—), 1,4-butyl (—CH₂CH₂CH₂CH₂—), and the like.

“Alkylene” refers to a straight or branched chain hydrocarbon radical, which is saturated, having from one to twelve carbon atoms (C₁₋₁₂alkylene), in certain embodiments one to eight carbon atoms (C₁₋₈alkylene) or one to six carbon atoms (C₁₋₆alkylene), or one to four carbon atoms (C₁₋₄alkylene), and which is divalent. Examples include methylene (—CH₂—), ethylene (—CH₂CH₂—), propylene (—CH₂CH₂CH₂—), 1-methylethylene (—CH(CH₃)CH₂—), butylene (—CH₂CH₂CH₂CH₂—), 1-methylpropylene (—CH(CH₃)CH₂CH₂—), 1,1-dimethylethylene (—C(CH₃)₂CH₂—), and 1,2-dimethylethylene (—CH(CH₃)CH(CH₃)—). Unless stated otherwise, the definitions propylene and butylene include all the possible isomeric forms of the groups in question with the same number of carbons. Thus, for example, propylene also includes 1-methylethylene and butylene includes 1-methylpropylene, 1,1-dimethylethylene, and 1,2-dimethylethylene.

“Alkynyl” is a straight or branched hydrocarbon containing normal, secondary or tertiary carbon atoms with at least one site of unsaturation, i.e., a carbon-carbon, sp triple bond. For example, an alkynyl group can have 2 to 20 carbon atoms (i.e., C₂-C₂₀ alkynyl), 2 to 8 carbon atoms (i.e., C₂-C₈ alkyne), or 2 to 6 carbon atoms (i.e., C₂-C₆ alkynyl). Examples of suitable alkynyl groups include, but are not limited to, acetylenic (—C≡CH), propargyl (—CH₂C≡CH), and the like.

The term “(C₂₋ₙ)alkynyl”, as used herein, wherein n is an integer, either alone or in combination with another radical, is intended to mean an unsaturated, acyclic straight or branched chain radical containing two to n carbon atoms, at least two of which are bonded to each other by a triple bond. Examples of such radicals include, but are not limited to, ethynyl, 1-propynyl, 2-propynyl, and 1-butynyl. When a (C₂₋ₙ)alkynyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “(C₃₋ₘ)cycloalkyl” as used herein, wherein m is an integer, either alone or in combination with another radical, is intended to mean a cycloalkyl substituent containing from 3 to m carbon atoms and includes, but is not limited to, cyclopropyl, cyclobutyl, cyclopentyl, cyclohexyl, cycloheptyl, and cyclooctyl.

The term “(C₃₋ₘ)cycloalkyl-(C₁₋ₙ)alkyl-” as used herein, wherein n and m are both integers, either alone or in combination with another radical, is intended to mean an alkyl radical having 1 to n carbon atoms as defined above which is itself substituted with a cycloalkyl radical containing from 3 to m carbon atoms as defined above. Examples of (C₃₋₇)cycloalkyl-(C₁₋₆)alkyl-include, but are not limited to, cyclopropylmethyl, cyclobutylmethyl, cyclopentylmethyl, cyclohexylmethyl, 1-cyclopropylethyl, 2-cyclopropylethyl, 1-cyclobutylethyl, 2-cyclobutylethyl, 1-cyclopentylethyl, 2-cyclopentylethyl, 1-cyclohexylethyl and 2-cyclohexylethyl. When a (C₃₋ₘ)cycloalkyl-(C₁₋ₙ)alkyl-group is substituted, it is understood that substituents may be attached to either the cycloalkyl or the alkyl portion thereof or both, unless specified otherwise, such that the substitution would give rise to a chemically stable compound, such as are recognized by those skilled in the art.

The term “halo” or “halogen” as used herein refers to fluoro (F), chloro (Cl), bromo (Br) and iodo (I).

The term “antiviral agent” as used herein is intended to mean an agent (compound or biological) that is effective to inhibit the formation and/or replication of a virus in a subject, for example, a human, including but not limited to agents that interfere with either host or viral mechanisms necessary for the formation and/or replication of a virus in a subject, for example, a human.

The term “aryl” as used herein refers to a single aromatic ring or a bicyclic or multicyclic ring. For example, an aryl group can have 6 to 20 carbon atoms, 6 to 14 carbon atoms, or 6 to 12 carbon atoms. Aryl includes a phenyl radical or an ortho-fused bicyclic or multicyclic radical having about 9 to 14 atoms in which at least one ring is aromatic (e.g., an aryl fused to one or more aryl or carbocycle). Such bicyclic or multicyclic rings may be optionally substituted with one or more (e.g., 1, 2 or 3) oxo groups on any carbocycle portion of the bicyclic or multicyclic ring. It is to be understood that the point of attachment of a bicyclic or multicyclic radical, as defined above, can be at any position of the ring including an aryl or a carbocycle portion of the ring. Typical aryl groups include, but are not limited to, phenyl, indenyl, naphthyl, 1, 2, 3, 4-tetrahydronaphthyl, anthracenyl, and the like. The term “aryl” as used herein, either alone or in combination with another radical, is intended to mean a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, indenyl, 1-naphthyl, 2-naphthyl, tetrahydronaphthyl and dihydronaphthyl.

As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

A prefix such as “Cᵤ₋ᵥ” or “(Cᵤ-Cᵥ)” indicates that the following group has from u to v carbon atoms. For example, “C₁₋₆alkyl” indicates that the alkyl group has from one to six carbon atoms.

As used herein, the term “dosage form” refers to a physically discrete unit of an active agent (e.g., a therapeutic, prophylactic, or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a prophylactic or therapeutic dosing regimen). Those of ordinary skill in the art appreciate that the total amount of a composition or agent administered to a particular subject is determined by one or more attending physicians and may involve administration of multiple dosage forms.

“Hydroxy” or “hydroxyl” refers to the —OH radical.

The term “inhibitor of HIV replication” as used herein is intended to mean an agent capable of reducing or eliminating the ability of HIV to replicate in a host cell, whether in vitro, ex vivo, or in vivo.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event or circumstances may or may not occur, and that the description includes instances where said event or circumstance occurs and instances in which it does not. For example, “optionally substituted heterocyclyl” means that the heterocyclyl radical may or may not be substituted and that the description includes both substituted heterocyclyl radicals and heterocyclyl radicals having no substitution.

“Oxo” refers to the ═O substituent.

A “pharmaceutical composition” refers to a formulation of a compound of the embodiments disclosed herein and a medium generally accepted in the art for the delivery of the biologically active compound to mammals, e.g., humans. Such a medium includes all pharmaceutically acceptable excipients.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or other pharmacologically inactive substance that is formulated in combination with a pharmacologically active ingredient of a pharmaceutical composition and is compatible with the other ingredients of the formulation and suitable for use in humans or domestic animals without undue toxicity, irritation, allergic response, and the like.

The term “pharmaceutically acceptable salt,” as used herein, refers to salts of such compounds that are appropriate for use in pharmaceutical contexts, i.e., salts which are, within the scope of sound medical judgment, suitable for use in contact with the tissues of humans and/or animals without undue toxicity, irritation, allergic response and the like, and are commensurate with a reasonable benefit/risk ratio. Pharmaceutically acceptable salts are well known in the art. For example, S. M. Berge et al. describe several pharmaceutically acceptable salts in detail in the Journal of Pharmaceutical Sciences. S. M. Berge et al., J. Pharma. Sci., 66:119 (1977).

Examples of “pharmaceutically acceptable salts” of the compounds disclosed herein include salts derived from an appropriate base, such as an alkali metal (for example, sodium), an alkaline earth metal (for example, magnesium), ammonium and NX₄⁺ (wherein X is C₁₋₄alkyl). Pharmaceutically acceptable salts of a nitrogen atom or an amino group include, for example, salts of organic carboxylic acids such as acetic, trifluoroacetic, adipic, ascorbic, aspartic, butyric, camphoric, cinnamic, citric, digluconic, glutamic, glycolic, glycerophosphoric, formic, hexanoic, benzoic, lactic, fumaric, tartaric, maleic, hydroxymaleic, malonic, malic, mandelic, isethionic, lactobionic, nicotinic, oxalic, pamoic, pectinic, phenylacetic, 3-phenylpropionic, pivalic, propionic, pyruvic, salicylic, stearic, sulfanilic, tartaric, undecanoic, and succinic acids; organic sulfonic acids, such as methanesulfonic, ethanesulfonic, camphorsulfonic, mesitylenesulfonic, benzenesulfonic, p-toluenesulfonic acids, naphthalenesulfonic, and 2-naphthalenesulfonic; and inorganic acids, such as hydrochloric, hydrobromic, sulfuric, phosphoric, nitric, and sulfamic acids. Pharmaceutically acceptable salts of a compound of a hydroxy group include the anion of said compound in combination with a suitable cation such as Na⁺ and NX₄⁺ (wherein X is independently selected from H and a C₁₋₄alkyl group).

For therapeutic use, salts of active ingredients of the compounds disclosed herein will typically be pharmaceutically acceptable, i.e., they will be salts derived from a physiologically acceptable acid or base. However, salts of acids or bases which are not pharmaceutically acceptable may also find use, for example, in the preparation or purification of a compound of formula I or another compound of the embodiments disclosed herein. All salts, whether or not derived from a physiologically acceptable acid or base, are within the scope of the embodiments disclosed herein.

Metal salts typically are prepared by reacting the metal hydroxide with a compound according to the embodiments disclosed herein. Examples of metal salts which are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄ or organic sulfonic acids, to basic centers, typically amines. Finally, it is to be understood that the compositions herein comprise compounds disclosed herein in their un-ionized, as well as zwitterionic form.

“Prevention” or “preventing” means any treatment of a disease or condition that causes the clinical symptoms of the disease or condition not to develop. Compositions may, in some embodiments, be administered to a subject (including a human) who is at risk of having the disease or condition. As used herein, the terms “preventing” and “prevention” encompass the administration of a compound, composition, or pharmaceutically acceptable salt according to the embodiments disclosed herein pre- or post-exposure of the individual to HIV, but before the appearance of symptoms of HIV infection, and/or prior to the detection of the virus in the blood. The terms also refer to prevention of the appearance of symptoms of the disease and/or to prevent the virus from reaching detectible levels in the blood. The terms include both pre-exposure prophylaxis (PrEP), as well as post-exposure prophylaxis (PEP) and event driven or “on demand” prophylaxis. The terms also refer to prevention of perinatal transmission of HIV from mother to baby, by administration to the mother before giving birth and to the child within the first days of life. The terms also refer to prevention of transmission of HIV through blood transfusion.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another. In any of the embodiments disclosed herein, compounds disclosed herein may be in the form of a stereoisomer thereof.

As used herein, the term “subject” refers to an organism, typically a mammal (e.g., a human). In some embodiments, a human subject is an adult, adolescent, or pediatric subject. In some embodiments, a subject is suffering from a relevant disease or condition. In some embodiments, a subject is susceptible to a disease or condition. In some embodiments, a subject displays one or more symptoms or characteristics of a disease or condition. In some embodiments, a subject does not display any symptom or characteristic of a disease or condition. In some embodiments, a subject is someone with one or more features characteristic of susceptibility to or risk of a disease or condition. In some embodiments, a subject is a patient. In some embodiments, a subject is an individual to whom diagnosis and/or therapy and/or prophylaxis is and/or has been administered.

As used herein, “therapeutically effective amount” is an amount that produces the desired effect for which it is administered. In some embodiments, the term “therapeutically effective amount” or “therapeutically effective dose” means an amount that is sufficient, when administered to a population suffering from or susceptible to a disease or condition in accordance with a therapeutic dosing regimen, to treat the disease or condition. In some embodiments, a therapeutically effective amount is one that reduces the incidence and/or severity of, stabilizes one or more characteristics of, and/or delays onset of, one or more symptoms of the disease or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease or condition) or fluids (e.g., blood, saliva, serum, sweat, tears, urine, etc.). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival. As used herein, the terms “treatment” and “treating” encompass the administration of a compound, composition, or pharmaceutically acceptable salt according to the embodiments disclosed herein to alleviate or eliminate symptoms of HIV infection, and/or to reduce viral load in a subject in need thereof. In some embodiments, a subject is a human patient.

The embodiments disclosed herein are also meant to encompass all pharmaceutically acceptable compounds of formula I being isotopically labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. In certain embodiments, these radiolabeled compounds are useful to help determine or measure the effectiveness of the compounds, by characterizing, for example, the site or mode of action, or binding affinity to pharmacologically important site of action. Certain isotopically labeled compounds of formula I, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

In certain embodiments, substitution with heavier isotopes such as deuterium, i.e., 2H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O, and ¹³N, can be useful in Positron Emission Topography (PET) studies for examining substrate receptor occupancy. Isotopically labeled compounds of formula I can be prepared by techniques known to those skilled in the art or by processes analogous to those described in the Examples as set out below using an appropriate isotopically labeled reagent in place of the non-labeled reagent previously employed.

The methods, compositions, kits and articles of manufacture provided herein use or include compounds (e.g., a compound of formula I) or pharmaceutically acceptable salts thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds increase resistance to metabolism, and thus are useful for increasing the half-life of compounds or pharmaceutically acceptable salts thereof, when administered to a mammal. See, e.g., Allen B. Foster, Deuterium Isotope Effects in Studies of Drug Metabolism, 5 Trends Pharmacol. Sci. 524, 524-27 (1984). Such compounds can be synthesized by means known in the art, for example by employing starting materials in which one or more hydrogen atoms have been replaced by deuterium.

The embodiments disclosed herein are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the embodiments disclosed herein include compounds produced by a process comprising administering a compound according to the embodiments disclosed herein to a mammal for a period of time sufficient to yield a metabolic product thereof. Such products are typically identified by administering a radiolabeled compound according to the embodiments disclosed herein in a detectable dose to an animal, such as rat, mouse, guinea pig, monkey, or to human, allowing sufficient time for metabolism to occur, and isolating its conversion products from the urine, blood or other biological samples.

The compounds of the embodiments disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic, scalemic, and optically pure forms. Optically active (+) and (−), (R)- and (S)-, or (D)- and (L)-isomers may be prepared using chiral synthons or chiral reagents, or resolved using methods such as chromatography and fractional crystallization. Techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

### Compounds

Provided herein are compounds that function as anti-HIV agents, pharmaceutical compositions comprising such compounds, optionally in combination with one or more (e.g., two, three, or four) additional therapeutic agents, and methods of using such compounds and compositions. All compound embodiments described herein include any pharmaceutically acceptable salt, stereoisomer, or mixture of stereoisomers thereof.

In one embodiment, a compound of Formula (I), or a pharmaceutically acceptable salt thereof, is provided:

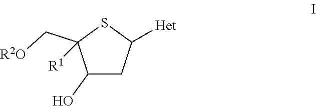

wherein

R¹ is C₂-C₈ alkynyl;

R² is selected from H,

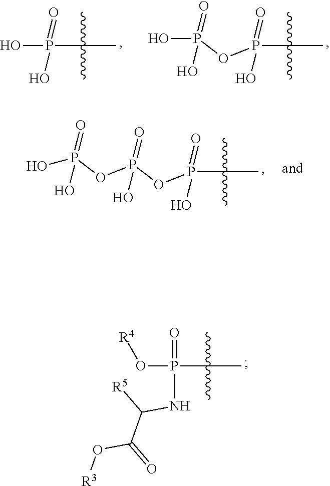

wherein:
R³ is C₁-C₈ alkyl;
R⁴ is selected from phenyl and naphthyl; and
R⁵ is H or C₁-C₆ alkyl; and

Het is

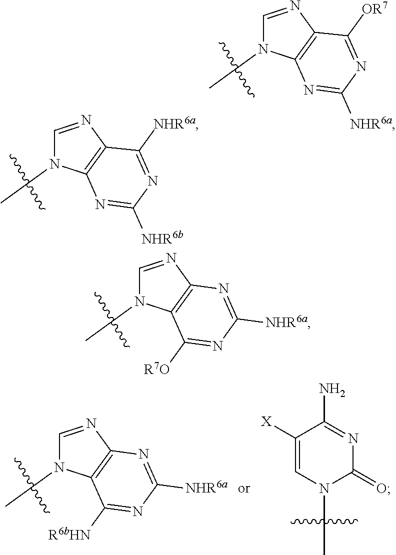

wherein:
X is halo;
R^{6a} is selected from H, C₁-C₆ alkyl, C₃-C₈ cycloalkyl, and C₁-C₆-alkylene-C₃-C₈ cycloalkyl;
R^{6b} is selected from H, C₁-C₆ alkyl, C₃-C₈ cycloalkyl, and C₁-C₆-alkylene-C₃-C₈ cycloalkyl; and
R⁷ is C₁-C₆ alkyl.

In some embodiments of the compound of formula I, or a pharmaceutically acceptable salt thereof, R¹ is C₂-C₆-alkynyl. In some embodiments, R¹ is C₂-C₄-alkynyl. In some embodiments, R¹ is C₂-C₃-alkynyl. In some embodiments, R¹ is

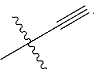 [SMILES: *C#C]

In some embodiments of the compound of formula I, or a pharmaceutically acceptable salt thereof, R² is H. In some embodiments, R² is selected from

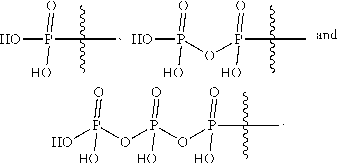

In some embodiments, R² is

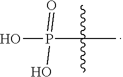 [SMILES: *P(=O)(O)O]

In some embodiments, R² is

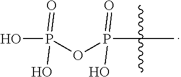 [SMILES: *P(=O)(O)OP(=O)(O)O]

In some embodiments, R² is

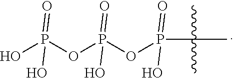

In some embodiments, R² is H or

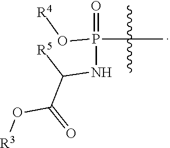

In some embodiments, R² is

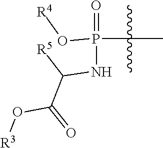

In some embodiments, R² is selected from

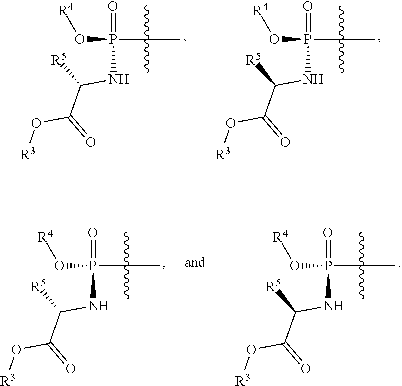

In some embodiments, R² is

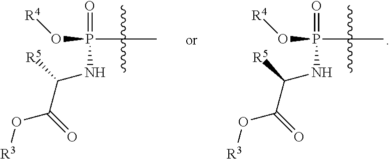

In some embodiments, R² is

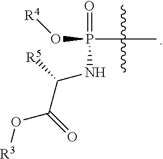

In some embodiments, R² is

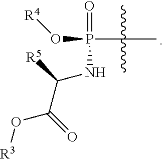

In some embodiments, R² is

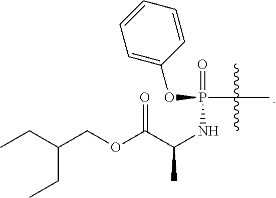 [SMILES: *[P@@](=O)(N[C@@H](C)C(=O)OCC(CC)CC)Oc1ccccc1]

In some embodiments of the compound of formula I, or a pharmaceutically acceptable salt thereof, R³ is C₁-C₆ alkyl. In some embodiments, R³ is C₁-C₄ alkyl. In some embodiments, R³ is a branched C₁-C₆ alkyl. In some embodiments, R³ is

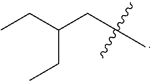 [SMILES: *CC(CC)CC]

In some embodiments of the compound of formula I, or a pharmaceutically acceptable salt thereof, R⁴ is phenyl. In some embodiments, R⁴ is naphthyl.

In some embodiments of the compound of formula I, or a pharmaceutically acceptable salt thereof, R⁵ is H or C₁-C₄ alkyl. In some embodiments, R⁵ is H. In some embodiments, R⁵ is C₁-C₃ alkyl. In some embodiments, R⁵ is C₁-C₂ alkyl. In some embodiments, R⁵ is CH₃.

In some embodiments of the compound of formula I, or a pharmaceutically acceptable salt thereof, Het is

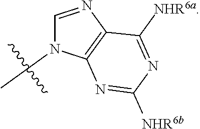

In some embodiments, Het is

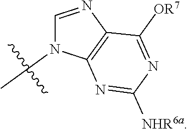

In some embodiments, Het is

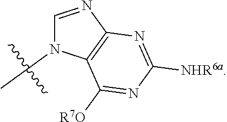

In some embodiments, Het is

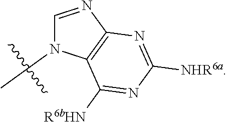

In some embodiments, Het is

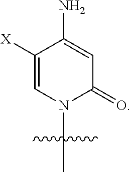

In some embodiments of the compound of formula I, or a pharmaceutically acceptable salt thereof, X is selected from F, Cl, and I. In some embodiments, X is F. In some embodiments, X is Cl. In some embodiments, X is I.

In some embodiments of the compound of formula I, or a pharmaceutically acceptable salt thereof, R^{6a} is H. In some embodiments, R^{6a} is C₁-C₆ alkyl. In some embodiments, R^{6a} is C₁-C₄ alkyl. In some embodiments, R^{6a} is C₃-C₈ cycloalkyl. In some embodiments, R^{6a} is C₃-C₆ cycloalkyl. In some embodiments, R^{6a} is C₃-C₄ cycloalkyl. In some embodiments R^{6a} is cyclopropyl. In some embodiments, R^{6a} is C₁-C₆-alkylene-C₃-C₈ cycloalkyl. In some embodiments, R^{6a} is C₁-C₄-alkylene-C₃-C₆ cycloalkyl. In some embodiments, R^{6a} is CH₂—C₃-C₄ cycloalkyl.

In some embodiments of the compound of formula I, or a pharmaceutically acceptable salt thereof, R^{6a} and R^{6b} are independently selected from H, C₁-C₆ alkyl, C₃-C₈ cycloalkyl, and C₁-C₆-alkylene-C₃-C₈ cycloalkyl. In some embodiments, R^{6a} and R^{6b} are independently selected from H, C₁-C₄ alkyl, C₃-C₆ cycloalkyl, C₁-C₄-alkylene-C₃-C₈ cycloalkyl, and C₁-C₄-alkylene-C₃-C₆ cycloalkyl. In some embodiments, R^{6a} and R^{6b} are independently selected from H, C₁-C₄ alkyl, C₃-C₄ cycloalkyl, and CH₂—C₃-C₄ cycloalkyl. In some embodiments, R^{6a} and R^{6b} are independently chosen from H, and cyclopropyl. In some embodiments, one of R^{6a} and R^{6b} is H and the other is cyclopropyl. In some embodiments, R^{6a} and R^{6b} are both H.

In some embodiments of the compound of formula I, or a pharmaceutically acceptable salt thereof, R^{6a} and R^{6b} are different. In some embodiments, R^{6a} and R^{6b} are the same.

In some embodiments of the compound of formula I, or a pharmaceutically acceptable salt thereof, R⁷ is C₁-C₄ alkyl. In some embodiments, R⁷ is ethyl.

In some embodiments of the compound of formula I, or a pharmaceutically acceptable salt thereof, Het is selected from

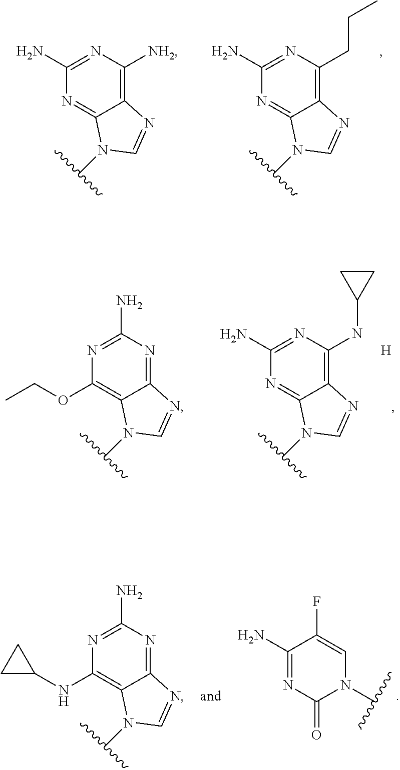

In some embodiments, the compound of formula I is a compound of formula (II):

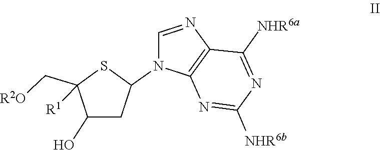

or a pharmaceutically acceptable salt thereof, wherein R¹, R², R^{6a}, and R^{6b} are as defined in formula I.

In some embodiments, the compound of formula I is a compound of formula (III):

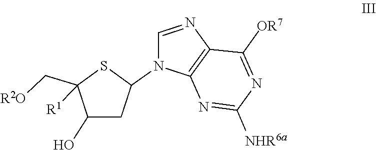

or a pharmaceutically acceptable salt thereof, wherein R¹, R², R^{6a}, and R⁷ are as defined in formula I.

In some embodiments, the compound of formula I is a compound of formula (IV):

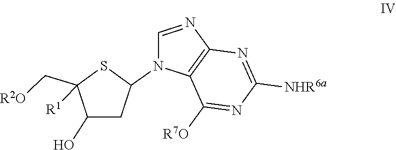

or a pharmaceutically acceptable salt thereof, wherein R¹, R², R^{6a}, and R⁷ are as defined in formula I.

In some embodiments, the compound of formula I is a compound of formula (V):

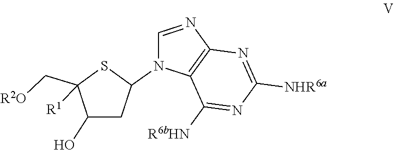

or a pharmaceutically acceptable salt thereof, wherein R¹, R², R^{6a}, and R^{6b} are as defined in formula I.

In some embodiments, the compound of formula I is a compound of formula (VI):

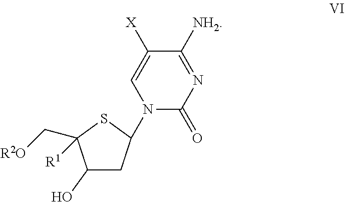

or a pharmaceutically acceptable salt thereof, wherein R¹, R², and X are as defined in formula I.

In some embodiments, the compound of formula I is a compound of formula (VII):

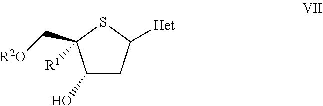

or a pharmaceutically acceptable salt thereof, wherein R¹, R², and Het are as defined in formula I.

In some embodiments, the compound of formula VII is a compound of formula (VIIa) or (VIIb):

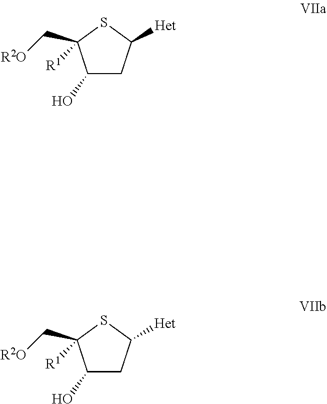

or a pharmaceutically acceptable salt thereof, wherein R¹, R², and Het are as defined in formula I.

In some embodiments of the compound of formula I, II, III, IV, V, VI, VII, VIIa, or VIIb, or a pharmaceutically acceptable salt thereof, R¹ is

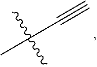 [SMILES: *C#C]

and R² is selected from H,

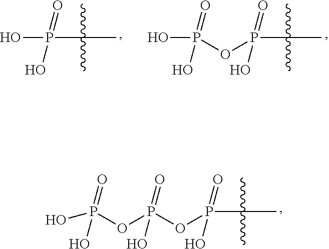 [SMILES: *P(=O)(O)O ; *P(=O)(O)OP(=O)(O)O ; *P(=O)(O)OP(=O)(O)OP(=O)(O)O]

In some embodiments, R¹ is

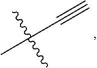 [SMILES: *C#C]

and R² is H. In some embodiments, R¹ is

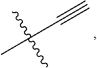 [SMILES: *C#C]

and R² is selected from

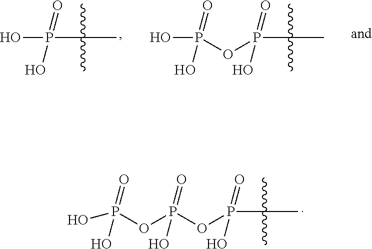 [SMILES: *P(=O)(O)O ; *P(=O)(O)OP(=O)(O)O ; *P(=O)(O)OP(=O)(O)OP(=O)(O)O]

In some embodiments, R¹ is

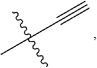 [SMILES: *C#C]

### and R² is

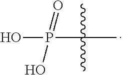 [SMILES: *P(=O)(O)O]

In some embodiments, R¹ is

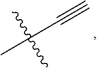 [SMILES: *C#C]

### and R² is

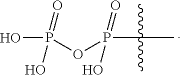 [SMILES: *P(=O)(O)OP(=O)(O)O]

In some embodiments, R¹ is

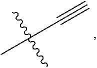 [SMILES: *C#C]

### and R² is

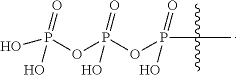 [SMILES: *P(=O)(O)OP(=O)(O)OP(=O)(O)O]

In some embodiments, R¹ is

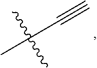 [SMILES: *C#C]

### and R² is H or

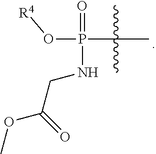

In some embodiments, R¹ is

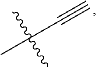 [SMILES: *C#C]

### and R² is

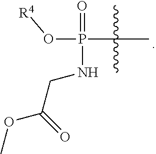

In some embodiments, R¹ is

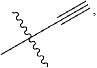 [SMILES: *C#C]

and R² is selected from

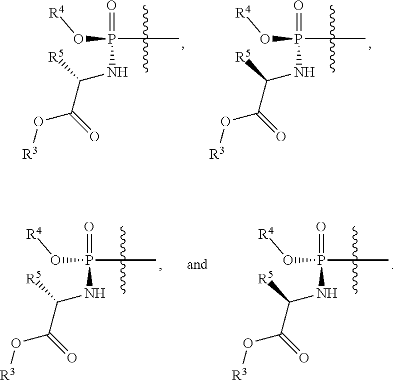

In some embodiments, the compound of formula VIIa is a compound of formula (VIII):

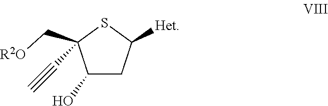

or a pharmaceutically acceptable salt thereof, wherein R² and Het are as defined in formula I.

In some embodiments, the compound of formula VIIb is a compound of formula (IX):

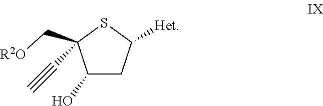

or a pharmaceutically acceptable salt thereof, wherein R² and Het are as defined in formula I.

In some embodiments, the compounds have the formula:

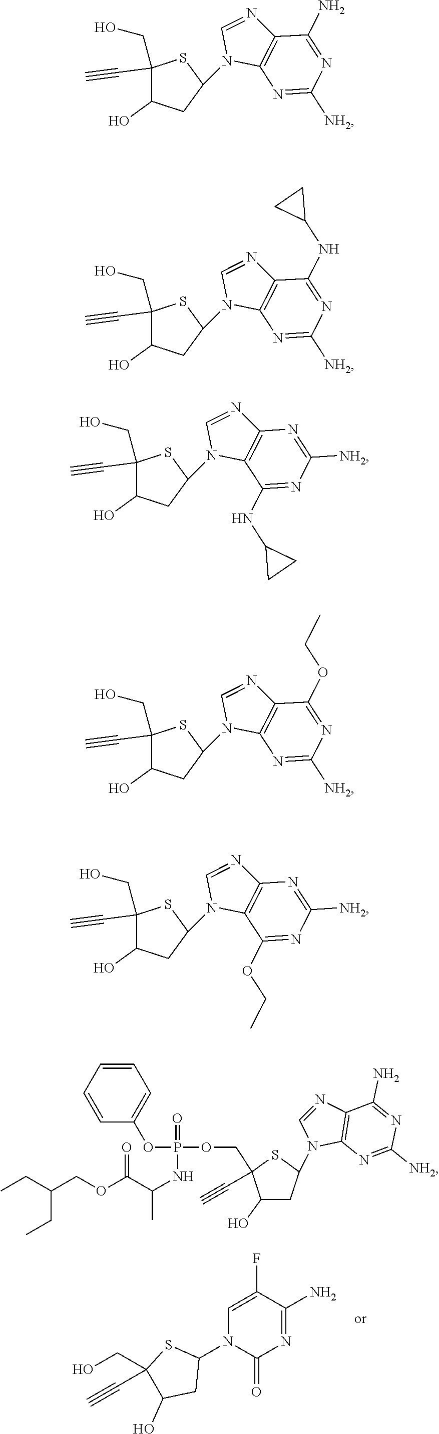

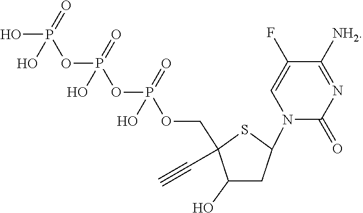 [SMILES: C#CC1(COP(=O)(O)OP(=O)(O)OP(=O)(O)O)SC(n2cc(F)c(N)nc2=O)CC1O]

or a pharmaceutically acceptable salt thereof.

In some embodiments, the compounds have the formula:

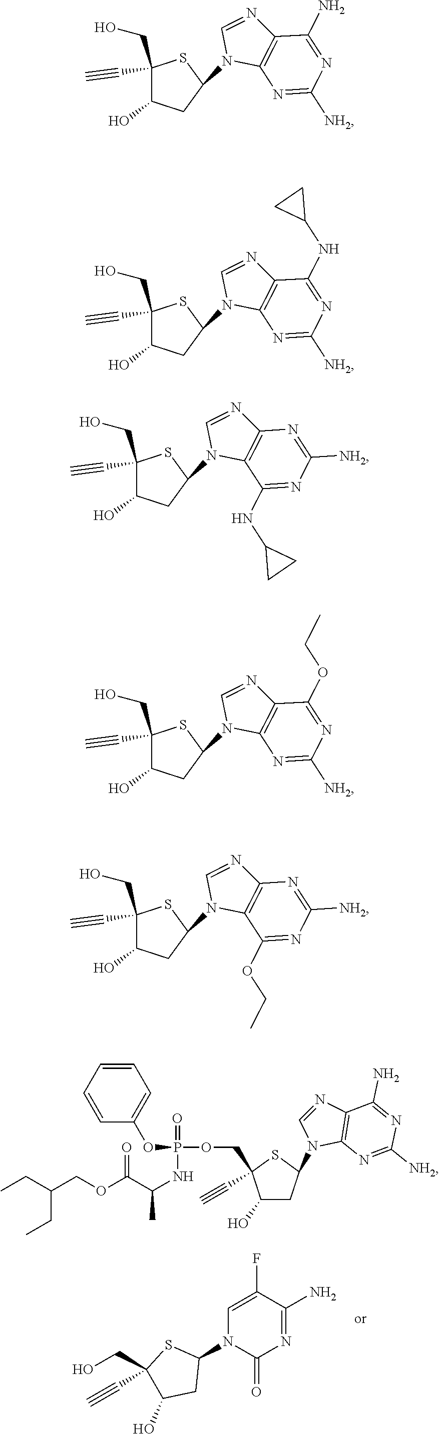

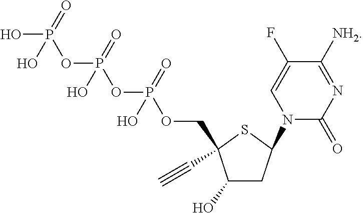 [SMILES: C#C[C@]1(COP(=O)(O)OP(=O)(O)OP(=O)(O)O)S[C@@H](n2cc(F)c(N)nc2=O)C[C@@H]1O]

or a pharmaceutically acceptable salt thereof.

In some embodiments, the compounds have the formula:

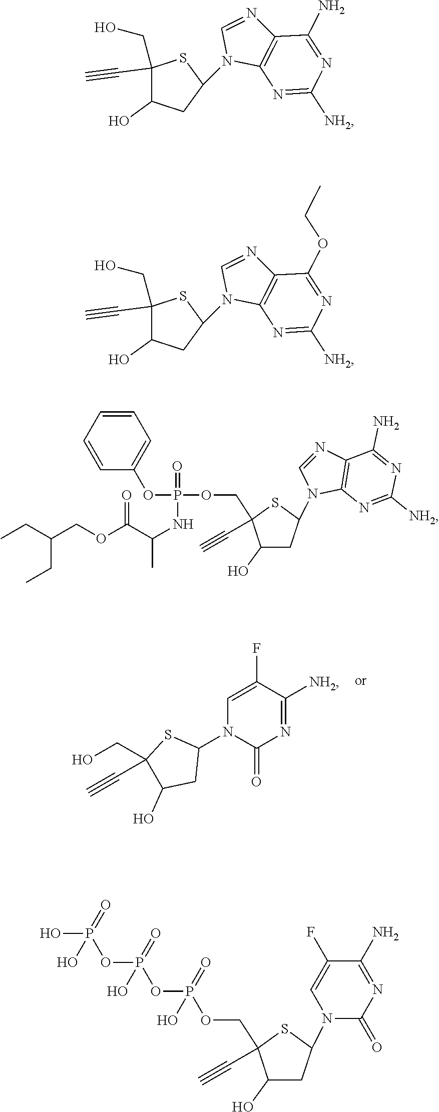 [SMILES: C#CC1(CO)SC(n2cnc3c(N)nc(N)nc32)CC1O ; C#CC1(CO)SC(n2cnc3c(OCC)nc(N)nc32)CC1O ; C#CC1(COP(=O)(NC(C)C(=O)OCC(CC)CC)Oc2ccccc2)SC(n2cnc3c(N)nc(N)nc32)CC1O ; C#CC1(CO)SC(n2cc(F)c(N)nc2=O)CC1O ; "or" ; C#CC1(COP(=O)(O)OP(=O)(O)OP(=O)(O)O)SC(n2cc(F)c(N)nc2=O)CC1O]

or a pharmaceutically acceptable salt thereof.

In some embodiments, the compounds have the formula:

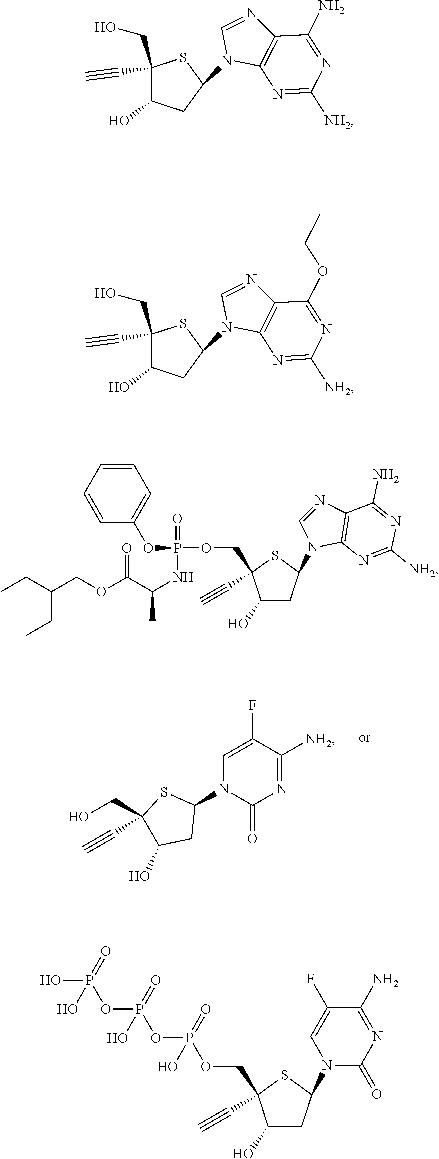 [SMILES: C#C[C@]1(CO)S[C@@H](n2cnc3c(N)nc(N)nc32)C[C@@H]1O ; C#C[C@]1(CO)S[C@@H](n2cnc3c(OCC)nc(N)nc32)C[C@@H]1O ; C#C[C@]1(CO[P@@](=O)(N[C@@H](C)C(=O)OCC(CC)CC)Oc2ccccc2)S[C@@H](n2cnc3c(N)nc(N)nc32)C[C@@H]1O ; C#C[C@]1(CO)S[C@@H](n2cc(F)c(N)nc2=O)C[C@@H]1O ; "or" ; C#C[C@]1(COP(=O)(O)OP(=O)(O)OP(=O)(O)O)S[C@@H](n2cc(F)c(N)nc2=O)C[C@@H]1O]

or a pharmaceutically acceptable salt thereof.

It is understood that any embodiment of the compounds of any one of formulas I, II, III, IV, V, VI, VII, VIIa, VIIb, VIII, and IX, as set forth above, and any specific group or substituent set forth herein (e.g., R¹, R², R³, R⁴, R⁵, R^{6a}, R^{6b}, R⁷, Het, and X, and substituents thereof) in the compounds of formulas I, II, III, IV, V, VI, VII, VIIa, VIIb, VIII, and IX, as set forth above, may be independently combined with other embodiments and/or substituents of compounds of any one of formulas I, II, III, IV, V, VI, VII, VIIa, VIIb, VIII, and IX, to form embodiments not specifically set forth above. In addition, in the event that a list of substituents are not listed for any particular R¹, R², R³, R⁴, R⁵, R^{6a}, R^{6b}, R⁷, Het, and X group in a particular embodiment and/or claim, it is understood that each individual substituent may be deleted from the particular embodiment and/or claim and that the remaining list of substituents will be considered to be within the scope of the embodiments disclosed herein.

The compounds of formulas I, II, III, IV, V, VI, VII, VIIa, VIIb, VIII, and IX, or pharmaceutically acceptable salts thereof, for example, compounds of formulas I, II, III, IV, V, VI, VII, VIIa, VIIb, VIII, and IX wherein R² is H or —P(O)(OR⁴)NHCH(R⁵)CO₂R³ or a pharmaceutically acceptable salts thereof, may be converted intracellularly in vivo by one or more mechanisms (e.g., enzyme-catalyzed chemical reactions) to the corresponding 5′ triphosphate which can inhibit the HIV reverse transcriptase enzyme and result in antiviral activity after administration of a compound of formulas I, II, III, IV, V, VI, VII, VIIa, VIIb, VIII, and IX, or a pharmaceutically acceptable salt thereof, to a subject. In some embodiments, the compounds of formulas I, II, III, IV, V, VI, VII, VIIa, VIIb, VIII, and IX, or pharmaceutically acceptable salts thereof, selectively inhibit the HIV reverse transcriptase enzyme over one or more host DNA polymerases. In some embodiments, the compounds of formulas I, II, III, IV, V, VI, VII, VIIa, VIIb, VIII, and IX, or pharmaceutically acceptable salts thereof, may have pharmacokinetic and/or other properties that make them amenable to less frequent administration.

### Pharmaceutical Compositions

In some embodiments, the present disclosure provides a pharmaceutical composition comprising a compound of the present disclosure, and a pharmaceutically acceptable excipient.

In some embodiments, the pharmaceutical composition comprises one, two, three, or four additional therapeutic agents, as more fully set forth below.

In some embodiments, a composition comprising a compound of the present disclosure, or a pharmaceutically acceptable salt thereof in one variation does not contain an agent that affects the rate at which the active ingredient is metabolized. Thus, it is understood that compositions comprising a compound of the present disclosure in one aspect do not comprise an agent that would affect (e.g., slow, hinder or retard) the metabolism of a compound of the present disclosure or any other active ingredient administered separately, sequentially or simultaneously with a compound of the present disclosure. It is also understood that any of the methods, kits, articles of manufacture and the like detailed herein in one aspect do not comprise an agent that would affect (e.g., slow, hinder or retard) the metabolism of a compound of the present disclosure or any other active ingredient administered separately, sequentially or simultaneously with a compound of the present disclosure.

In some embodiments, the pharmaceutical compositions described above are for use in a human or an animal.

The disclosure further includes a compound of the present disclosure for administration as a single active ingredient of a pharmaceutically acceptable composition that can be prepared by methods known in the art, for example by binding the active ingredient to a pharmaceutically acceptable, therapeutically inert organic and/or inorganic carrier or excipient, or by mixing therewith.

In one aspect, provided herein is the use of a compound of the present disclosure as a second or other active ingredient having a synergistic effect with other active ingredients in known drugs, or administration of the compound of the present disclosure together with such drugs.

### Methods of Treatment

### HIV Infection

The present disclosure provides methods of treating and/or preventing human immunodeficiency virus (HIV) infection in a subject. In some embodiments, a method of treating and/or preventing HIV infection in a subject comprises administering to the subject a composition provided herein. In some embodiments, the method is for treating and/or preventing HIV-1 infection. In some embodiments, the method is for treating and/or preventing HIV-2 infection.

In some embodiments, a method of treating HIV infection in a subject in need thereof comprises administering to the subject a composition provided herein. In some such embodiments, the subject is HIV positive. In some such embodiments, the subject is of unknown HIV status. In some such embodiments, the subject is not HIV negative.

In some embodiments, a method of preventing HIV infection in a subject at risk thereof comprises administering to the subject a composition provided herein. In some such embodiments, the subject is HIV negative. In some embodiments, the subject is at risk of acquiring HIV infection.

In some embodiments, provided compositions are combined with one, two, three, or four additional therapeutic agents selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, provided compositions are combined with one, two, three, or four additional therapeutic agents selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, provided compositions are combined with one, two, three, or four additional therapeutic agents selected from dolutegravir, cabotegravir, islatravir, darunavir, bictegravir, elsulfavirine, rilpivirine, and lenacapavir, and combinations thereof.

### HIV Combination Therapy

In certain embodiments, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents. In one embodiment, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents.

In one embodiment, pharmaceutical compositions comprising a compound disclosed herein, or a pharmaceutically acceptable salt thereof, in combination with one, two, three, or four additional therapeutic agents, and a pharmaceutically acceptable carrier, diluent, or excipient are provided.

In certain embodiments, the present disclosure provides a method for treating an HIV infection, comprising administering to a subject in need thereof a therapeutically effective amount of a compound disclosed herein, or a pharmaceutically acceptable salt thereof, in combination with a therapeutically effective amount of one, two, three, or four additional therapeutic agents which are suitable for treating an HIV infection.

In certain embodiments, a compound disclosed herein, or a pharmaceutically acceptable salt thereof, is combined with one, two, three, four, or more additional therapeutic agents. In certain embodiments, a compound disclosed herein, or a pharmaceutically acceptable salt thereof, is combined with one, two, three, or four additional therapeutic agents. In certain embodiments, a compound disclosed herein, or a pharmaceutically acceptable salt thereof, is combined with two additional therapeutic agents. In other embodiments, a compound disclosed herein, or a pharmaceutically acceptable salt thereof, is combined with three additional therapeutic agents. In further embodiments, a compound disclosed herein, or a pharmaceutically acceptable salt thereof, is combined with four additional therapeutic agents. The one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a compound disclosed herein is administered with one, two, three, or four additional therapeutic agents. Co-administration of a compound disclosed herein with one, two, three, or four additional therapeutic agents generally refers to simultaneous or sequential administration of a compound disclosed herein and one, two, three, or four additional therapeutic agents, such that therapeutically effective amounts of the compound disclosed herein and the one, two, three, or four additional therapeutic agents are both present in the body of the patient. When administered sequentially, the combination may be administered in two or more administrations.

Co-administration includes administration of unit dosages of the compounds disclosed herein before or after administration of unit dosages of one, two, three, or four additional therapeutic agents. For example, the compound disclosed herein may be administered within seconds, minutes, or hours of the administration of the one, two, three, or four additional therapeutic agents. In some embodiments, a unit dose of a compound disclosed herein is administered first, followed within seconds or minutes by administration of a unit dose of one, two, three, or four additional therapeutic agents. Alternatively, a unit dose of one, two, three, or four additional therapeutic agents is administered first, followed by administration of a unit dose of a compound disclosed herein within seconds or minutes. In other embodiments, a unit dose of a compound disclosed herein is administered first, followed, after a period of hours (e.g., 1-12 hours), by administration of a unit dose of one, two, three, or four additional therapeutic agents. In yet other embodiments, a unit dose of one, two, three, or four additional therapeutic agents is administered first, followed, after a period of hours (e.g., 1-12 hours), by administration of a unit dose of a compound disclosed herein.

In certain embodiments, a kit comprising a compound disclosed herein (e.g., a compound of formula I, II, III, IV, V, VI, VII, VIIa, VIIb, VIII, or IX), or a pharmaceutically acceptable salt thereof, in combination with one or more (e.g., one, two, three, or four) additional therapeutic agents is provided.

In a specific embodiment, the kit includes a compound disclosed herein, or a pharmaceutically acceptable salt thereof, an HIV nucleoside or nucleotide inhibitor of reverse transcriptase and an HIV capsid inhibitor or an HIV capsid polymerization inhibitor.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are chosen from dolutegravir, cabotegravir, islatravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir and lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; PREZCOBIX® (darunavir and cobicistat); dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, ACC-008 (ACC-007+lamivudine+tenofovir disoproxil fumarate), VM-1500+emtricitabine+tenofovir disoproxil, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, (elsulfavirine, VM-1500), VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, Gamimune, metenkefalin, naltrexone, Prolastin, REP 9, VSSP, Hlviral, SB-728-T, 1,5-dicaffeoylquinic acid, rHIV7-shl-TAR-CCR5RZ, AAV-eCD4-Ig gene therapy, MazF gene therapy, BlockAide, bevirimat, ABBV-382, obefazimod (ABX-464), AG-1105, APH-0812, APH0202, bryostatin-1, bryostatin-23, bryostatin analogs, SUW-133, BIT-225, BRII-732, BRII-778, Codivir, CYT-107, CS-TATI-1, fluoro-beta-D-arabinose nucleic acid (FANA)-modified antisense oligonucleotides, FX-101, griffithsin, HGTV-43, HPH-116, HRS-5685, HivCide-I, hydroxychloroquine, IMB-10035, IMO-3100, IND-02, JL-18008, LADAVRU, LLDT-8, MK-1376, MK-2048, MK-4250, MK-8507, MK-8558, islatravir (MK-8591), NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-C₀₄₈, ISR-48, ISR-49, MK-8527, cannabinoids, ENOB-HV-32, T-1144, VIR-576, nipamovir, Covimro, WP-1122, ZFP-362, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, elunonavir (GS-1156), TMB-657 (PPL-100), T-169, BL-008, MK-8122, TMB-607, GRL-02031, and TMC-310911.

Additional examples of HIV protease inhibitors are described, e.g., in U.S. Pat. No. 10,294,234, and U.S. Patent Application Publication Nos. US2020030327 and US2019210978.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, difluoro-biphenyl-diarylpyrimidines (DAPY), efavirenz, etravirine, GS-5894, lentinan, nevirapine, rilpivirine, ACC-007, ACC-018, AIC-292, F-18, KM-023, PC-1005, M1-TFV, M2-TFV, VM-1500A-LAI, PF-3450074, elsulfavirine (sustained release oral), doravirine+islatravir (fixed dose combination/oral tablet formulation), elsulfavirine (long acting injectable nanosuspension), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir amibufenamide fumarate (HS-10234), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, GSK-4023991, MK-8504, islatravir, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, US2022119426 and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, GS-1720, GS-6212, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, S-365598, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV capsid inhibitors include, but are not limited to, those described in U.S. Patent Application Publication Nos. US2014221356 and US2016016973.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, HIV-1 fusion inhibitors (P26-Bapc), ITV-1, ITV-2, ITV-3, ITV-4, CPT-31, Cl3hmAb, lipovirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, balixafortide and vMIP (Haimipu).

### HIV Maturation Inhibitors

Examples of HIV maturation inhibitors include, but are not limited to, BMS-955176, GSK-3640254, VH-3739937 (GSK-3739937), HRF-10071 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, xevinapant (AT-406, Debio-1143)), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, ixazomib, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-4182, GS-CA1, AVI-621, AVI-101, AVI-201, AVI-301, and AVI-CAN1-15 series, PF-3450074, VH-4004280, VH-4011499, and compounds described in (GSK WO2019/087016).

Additional examples of capsid inhibitors include, but not limited to, those described in U.S. Patent Application Publication Nos. US2018051005 and US2016108030.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSFi4 (HVEML); CD272 (B and T lymphocyte associated (BTLA)); TNFRSF17 (BCMA, CD269), TNFSF13B (BAFF); TNFRSF18 (GITR), TNFSFi8 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAETIE; ULBP4); retinoic acid early transcript 1G (RAETIG; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSFi4 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSFi8 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, envafolimab (ASC-22, KN-035), PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, budigalimab (ABBV-181), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSFlA (NCBI Gene ID: 7132), TNFRSFlB (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRiKE) include, but are not limited to, OXS-3550, HIV-TriKE, and CD16-IL-15-B7H3 TriKe.

### Indoleamine-pyrrole-2,3-dioxygenase (IDO1) inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, SHR-2150, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti-LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as nogapendekin alfa (ALT-803), NKTR-255, and hetIL-15, interleukin-15/Fc fusion protein, AM-0015, NIZ-985, SO-C₁₀₁, IL-15 Synthorin (pegylated Il-15), P-22339, and a IL-15-PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, and peginterferon alfa-2b.

Phosphatidylinositol 3-kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

alpha-4/beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, Immuglo, MB-66, clone 3 human monoclonal antibody targeting KLIC (HIV infection), GS-9721, GS-5423, GS-2872, BG-HIV, VRC-HIVMAB091-00-AB. Anti-CD4 antibodies such as ibalizumab, TMB-365.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, 1B2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acids Res., 42 (Database issue):Dl 133-9 (2014), Mascola et al., Immunol Rev., 254(1):225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue):Dl 133-9 (2014), Mascola et al., Immunol Rev., 254(1):225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, D1D2, 10-1074-LS, Cl3hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, GSK-3810109 (N6LS), N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM-1400LS, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation GS-8588, MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AdC6-HIVgp140, AE-298p, anti-CD40.Env-gp140 vaccine, Ad4-EnvCl50, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4×, Vacc-C₅, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C₅₅, GS-1983, GS-6708, TVI-HIV-1, Ad-4 (Ad4-env Clade C+Ad4-mGag), Paxvax, EN41-UGR7C, EN41-FPA2, ENOB-HV-11, ENOB-HV-12, exoVACC, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, MVA.tHIVconsv3, MVA.tHIVconsv4, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A, B, C, A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglbl2 neutralizing VRC-01 antibody-stimulating anti-CD4 vaccine, arenavirus vector-based vaccines (Vaxwave, TheraT), MVA-BN HIV-1 vaccine regimen, mRNA based vaccines, VPI-211, HIV ANTI-CD40.ENV GP140, HIV ANTI-CD40.HIV5PEP, multimeric HIV gp120 vaccine TBL-1203HI, CH505 TF chTrimer, CD40.HIVRI.Env vaccine, VRC-HIVRGP096-00-VP, Drep-HIV-PT-1, BG505 MD39.3 mRNA, BG505 MD39.3 gp151 CD4KO mRNA, BG505 MD39.3 gp151 mRNA, mRNA-1644, mRNA-1547, and mRNA-1574, and anti-HIV vaccines described in WO2021011544 and WO2022155258.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; EDURANT® (dolutegravir+rilpivirine); raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101, XVIR-TAT.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-Env duoCAR T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells.

In certain embodiments, the agents described herein are combined with IMC-M113V, a TCR bispecific having a TCR binding domain that targets a peptide derived from the Gag protein presented by HLA*A02 on the surface of HIV infected cells and a second antigen binding domain that targets CD3.

### B-Cell Therapy

In certain embodiments, a compound as described herein is combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

A compound as disclosed herein (e.g., any compound of formula I, II, III, IV, V, VI, VII, VIIa, VIIb, VIII or IX, or a pharmaceutically acceptable salt thereof) may be combined with one, two, three, or four additional therapeutic agents in any dosage amount of the compound of formula I, II, III, IV, V, VI, VII, VIIa, VIIb, VIII or IX, or a pharmaceutically acceptable salt thereof (e.g., from 1 mg to 500 mg of compound).

In one embodiment, kits comprising a compound disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, lenacapavir, cabotegravir, rilpivirine, any integrase LA, VM-1500 LAI, GS-1614, maraviroc (LAI), tenofovir implant, islatravir, islatravir implant, islatravir prodrug, doravirine, LYN-172, raltegravir, XVIR-120, GSK-3739937 (long-acting), and long acting dolutegravir, CABENUVA® (cabotegravir LA+rilpivirine LA), VOCABRIA® (cabotegravir LA), APRETUDE® (cabotegravir LAI), REKAMBYS® (rilpivirine LA).

### Administration

### Routes of Administration

The compound of formula I, II, III, IV, V, VI, VII, VIIa, VIIb, VIII or IX, or a pharmaceutically acceptable salt thereof, (also referred to herein as the active ingredient) can be administered by any route appropriate to the condition to be treated. Suitable routes include, but are not limited to, oral, rectal, nasal, topical (including buccal and sublingual), transdermal, vaginal and parenteral (including subcutaneous, intramuscular, intravenous, intradermal, intrathecal and epidural), and the like. It will be appreciated that a suitable route may vary with, for example, the condition of the recipient. In certain embodiments, the compounds disclosed can be dosed parenterally. In certain embodiments, the compounds disclosed can be dosed intravenous, subcutaneous, or intramuscular. In certain embodiments, the compounds disclosed are orally bioavailable and can be dosed orally.

In some embodiments, the compound of formula I, II, III, IV, V, VI, VII, VIIa, VIIb, VIII or IX, or a pharmaceutically acceptable salt thereof, is administered with a syringe suitable for administration of the compound. In some embodiments, the syringe is disposable. In some embodiments, the syringe is reusable. In some embodiments, the syringe is pre-filled with the compound of formula I, II, III, IV, V, VI, VII, VIIa, VIIb, VIII, or IX, or a pharmaceutically acceptable salt thereof.

In some embodiments, the compound of formula I, II, III, IV, V, VI, VII, VIIa, VIIb, VIII, or IX, or a pharmaceutically acceptable salt thereof, is administered with an auto-injector comprising a syringe. In some embodiments, the syringe is disposable. In some embodiments, the syringe is reusable. In some embodiments, the syringe is pre-filled with the compound formula I, II, III, IV, V, VI, VII, VIIa, VIIb, VIII, or IX, or a pharmaceutically acceptable salt thereof.

### Dosing Regimen

In some embodiments, the compound, such as a compound of formula I, II, III, IV, V, VI, VII, VIIa, VIIb, VIII, or IX, or a pharmaceutically acceptable salt thereof, is administered to a subject in accordance with an effective dosing regimen for a desired period of time or duration, such as at least about once a day, at least about once a week, at least about once a month, at least about once every 2 months, at least about once every 3 months, at least about once every 4 months, at least about once every 6 months, or at least about once every 12 months or longer. In some embodiments, the compound is administered on a daily or intermittent schedule. In some embodiments, the compound is administered on a weekly schedule. In some embodiments, the compound is administered on a monthly schedule. In some embodiments, the compound is administered every two months. In some embodiments, the compound is administered every three months. In some embodiments, the compound is administered every four months. In some embodiments, the compound is administered every five months. In some embodiments, the compound is administered every 6 months.

In some embodiments, the compound, such as a compound of formula I, II, III, IV, V, VI, VII, VIIa, VIIb, VIII, or IX, or a pharmaceutically acceptable salt thereof, is subcutaneously or intramuscularly administered to a subject at least about once a month. In some embodiments, the compound (e.g., a compound of formula I, II, III, IV, V, VI, VII, VIIa, VIIb, VIII, or IX, or a pharmaceutically acceptable salt thereof), is subcutaneously or intramuscularly administered to a subject at least about once every 2 months or at least about once every 3 months, or at least about once every 4 months, or at least about once every 6 months. In some embodiments, the compound (e.g., a compound of formula I, II, III, IV, V, VI, VII, VIIa, VIIb, VIII, or IX, or a pharmaceutically acceptable salt thereof), is subcutaneously administered to a subject at least about once a month. In some embodiments, the compound (e.g., a compound of formula I, II, III, IV, V, VI, VII, VIIa, VIIb, VIII, or IX, or a pharmaceutically acceptable salt thereof), is subcutaneously administered to a subject at least about once every 2 months. In some embodiments, the compound (e.g., a compound of formula I, II, III, IV, V, VI, VII, VIIa, VIIb, VIII, or IX, or a pharmaceutically acceptable salt thereof), is subcutaneously administered to a subject at least about once every 3 months.

In some embodiments, the dosage or dosing frequency of a compound of formula I, II, III, IV, V, VI, VII, VIIa, VIIb, VIII, or IX, or a pharmaceutically acceptable salt thereof, is adjusted over the course of the treatment, based on the judgment of the administering physician.

In some embodiments, a compound as disclosed herein (e.g., a compound of formula I, II, III, IV, V, VI, VII, VIIa, VIIb, VIII, or IX) or a pharmaceutically acceptable salt thereof, may be administered in a dosage amount that is effective. For example, the dosage amount can be from 1 mg to 1000 mg of compound.

In some embodiments, the methods disclosed herein comprise event-driven administration of the compound of formula I, II, III, IV, V, VI, VII, VIIa, VIIb, VIII, or IX, or a pharmaceutically acceptable salt thereof, to the subject.

As used herein, the terms “event-driven” and “event-driven administration” refer to administration of the compound of formula I, II, III, IV, V, VI, VII, VIIa, VIIb, VIII, or IX, or a pharmaceutically acceptable salt thereof, (1) prior to an event (e.g., 2 hours, 1 day, 2 days, 5 day, or 7 or more days prior to the event) that would expose the individual to HIV (or that would otherwise increase the individual's risk of acquiring HIV); and/or (2) during an event (or more than one recurring event) that would expose the individual to HIV (or that would otherwise increase the individual's risk of acquiring HIV); and/or (3) after an event (or after the final event in a series of recurring events) that would expose the individual to HIV (or that would otherwise increase the individual's risk of acquiring HIV). In some embodiments, the event driven administration is performed pre-exposure of the subject to the HIV. In some embodiments, the event driven administration is performed post-exposure of the subject to the HIV. In some embodiments, the event driven administration is performed pre-exposure of the subject to the HIV and post-exposure of the subject to the HIV.

In some embodiments, the compound of formula I, II, III, IV, V, VI, VII, VIIa, VIIb, VIII or IX, or a pharmaceutically acceptable salt thereof, is administered before exposure of the subject to the HIV.

An example of event driven dosing regimen includes administration of the compound of formula I, II, III, IV, V, VI, VII, VIIa, VIIb, VIII or IX, or a pharmaceutically acceptable salt thereof, within 24 to 2 hours prior to HIV exposure (e.g., first sexual activity with sex partner known to be HIV positive, including sexual intercourse), followed by administration of the compound of formula I, II, III, IV, V, VI, VII, VIIa, VIIb, VIII, or IX, or a pharmaceutically acceptable salt, every 24 hours during the period of exposure (e.g., sexual activity with sex partner known to be HIV positive), followed by a further administration of the compound of formula I, II, III, IV, V, VI, VII, VIIa, VIIb, VIII, or IX, or a pharmaceutically acceptable salt thereof, after the last exposure (e.g., sexual activity with sex partner known to be HIV positive), and one last administration of the compound of formula I, II, III, IV, V, VI, VII, VIIa, VIIb, VIII, or IX or a pharmaceutically acceptable salt thereof, 24 hours later.

A further example of an event driven dosing regimen includes administration of the compound of formula I, II, III, IV, V, VI, VII, VIIa, VIIb, VIII, or IX, or a pharmaceutically acceptable salt thereof, within 24 hours before HIV exposure (e.g., sexual activity with sex partner known to be HIV positive), then daily administration during the period of exposure (e.g., sexual activity with sex partner known to be HIV positive, including the last sexual intercourse), followed by a last administration approximately 24 hours later after the last exposure (which may be an increased dose, such as a double dose).

In certain embodiments, e.g., when administered as PrEP, the compound of formula I, II, III, IV, V, VI, VII, VIIa, VIIb, VIII, or IX, or a pharmaceutically acceptable salt thereof, is administered daily. In certain embodiments, e.g., when administered as event-driven PrEP, the compound of formula I, II, III, IV, V, VI, VII, VIIa, VIIb, VIII, or IX, or a pharmaceutically acceptable salt thereof, is administered 1 hour to 10 days, 1 hour to 7 days, 1 hour to 5 days, 1 to 72 hours, 1 to 48 hours, 1 to 24 hours, or 12 to 12 hours prior to an event that would increase the individual's risk of acquiring HIV (e.g., prior to sex or other exposure to the HIV virus). In some embodiments, the compound of formula I, II, III, IV, V, VI, VII, VIIa, VIIb, VIII, or IX, or a pharmaceutically acceptable salt thereof, is administered within 10 days, 7 days, 5 days, 72 hours, 60 hours, 48 hours, 24 hours, 12 hours, 9 hours, 6 hours, 4 hours, 3 hours, 2 hours, or 1 hour prior to an event that would increase the individual's risk of acquiring HIV (e.g., prior to sex or other exposure to the HIV virus). In certain embodiments, when the compound of formula I, II, III, IV, V, VI, VII, VIIa, VIIb, VIII, or IX, or a pharmaceutically acceptable salt thereof, is administered prior to an event (e.g., administered prior to the event) that would increase the individual's risk of acquiring HIV, it is administered daily prior to the event (e.g., sexual activity). In certain embodiments, when the compound of formula I, II, III, IV, V, VI, VII, VIIa, VIIb, VIII, or IX, or a pharmaceutically acceptable salt thereof, is administered prior to an event that would increase the individual's risk of acquiring HIV, it is administered one to three times prior to the event.

In some embodiments, e.g., when administered as part of an event-driven PrEP regimen, the compound of formula I, II, III, IV, V, VI, VII, VIIa, VIIb, VIII, or IX, or a pharmaceutically acceptable salt thereof, is administered during the time of HIV-exposure. In certain embodiments wherein the compound of formula I, II, III, IV, V, VI, VII, VIIa, VIIb, VIII, or IX, is administered before exposure, the compound of formula I, II, III, IV, V, VI, VII, VIIa, VIIb, VIII, or IX, or a pharmaceutically acceptable salt thereof, is administered daily (e.g., as a single dose) during the time of HIV-exposure (e.g., during the time period of sexual activity with sex partner known to be HIV positive). In some embodiments, the compound of formula I, II, III, IV, V, VI, VII, VIIa, VIIb, VIII, or IX, or a pharmaceutically acceptable salt thereof, is administered daily (e.g., for 1 to 7 days) after final exposure to the HIV (e.g., after a period of sexual activity with sex partner known to be HIV positive). In some embodiments, the administration is continued for 1 or 2 days after final exposure to HIV.

Additional examples of PrEP and/or PEP can be found, for example, at the clinical trial summary titled “On Demand Antiretroviral Pre-exposure Prophylaxis for HIV Infection in Men Who Have Sex With Men” (Clinical Trial #NCT01473472); the clinical trial summary titled “Prevention of HIV in Île-de-France” (Clinical Trials #NCT03113123), and at Molina, et al. N. Engl. J. Med. 2015, 353:2237-2246, the disclosure of each of which is incorporated herein by reference in its entirety.

In some embodiments, methods for reducing the risk of acquiring HIV (e.g., HIV-1 and/or HIV-2) comprise administration of the compound of formula I, II, III, IV, V, VI, VII, VIIa, VIIb, VIII, or IX, or a pharmaceutically acceptable salt thereof, in combination with safer sex practices. In certain embodiments, methods for reducing the risk of acquiring HIV (e.g., HIV-1 and/or HIV-2) comprise administration to an individual at risk of acquiring HIV. Examples of individuals at high risk for acquiring HIV include, without limitation, an individual who is at risk of sexual transmission of HIV.

In some embodiments, the reduction in risk of acquiring HIV is at least about 40%, at least about 50%, at least about 60%, at least about 70%, at least about 80%, at least about 90%, or at least about 95%. In some embodiments, the reduction in risk of acquiring HIV is at least about 75%. In some embodiments, the reduction in risk of acquiring HIV is about 80%, about 85%, or about 90%.

### Formulation

Formulations suitable for parenteral administration include, but are not limited to, aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. In certain embodiments the suspension is a microsuspension. In certain embodiments the suspension is a nanosuspension.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and may be found, e.g., in Handbook of Pharmaceutical Excipients (eds. Rowe, Sheskey & Quinn), 6th edition 2009.

Formulations suitable for oral administration include, but are not limited to, solid preparations (e.g., powders, pills, capsules, and tablets) and liquid preparations (e.g., suspensions, syrups, elixirs, and the like), each of which may optionally contain one or more other compounds useful for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, the formulation is provided as a single dose, once-daily, or less frequently such as once weekly or once monthly, twice yearly or once yearly. Furthermore, the compound may be formulated for immediate or modified release such as extended or controlled release.

In certain embodiments, the active ingredient (e.g., a compound of formula I, II, III, IV, V, VI, VII, VIIa, VIIb, VIII, or IX) is present as a free base, zwitterion, or salt.

In certain embodiments the pharmaceutical composition disclosed herein is a parenteral formulation. In certain embodiments, the formulation is administered subcutaneously to a subject. In certain embodiments, the formulation is administered intramuscularly to a subject.

The amount of active ingredient that may be combined with the inactive ingredients to produce a dosage form may vary depending upon the intended treatment subject and the particular mode of administration. For example, in some embodiments, a dosage form for oral administration to humans may contain approximately 1 mg to 1000 mg of active material formulated with an appropriate and convenient amount of carrier material (e.g., inactive ingredient or excipient material). In certain embodiments, the carrier material varies from about 5% to about 95% of the total compositions (weight: weight or wt:wt).

It should be understood that in addition to the ingredients particularly mentioned above the compositions of these embodiments may include other agents in the art having regard to the type of composition in question, for example those suitable for oral administration may include flavoring agents.

### Kits and Articles of Manufacture

Kits that comprise a compound of the present disclosure, or an enantiomer, or pharmaceutically acceptable salt thereof, or a pharmaceutical composition containing any of the above, are also included in the present disclosure. In one embodiment, a kit further includes instructions for use. In one aspect, a kit includes a compound of the disclosure, or a pharmaceutically acceptable salt, tautomer, stereoisomer, mixture of stereoisomers, prodrug, or deuterated analog thereof, and a label and/or instructions for use of the compounds in the treatment of the indications, such as the diseases or conditions, described herein. In one embodiment, kits comprising a compound of the present disclosure, or a pharmaceutically acceptable salt thereof, in combination with one or more (e.g., one, two, three, four, one or two, or one to three, or one to four) additional therapeutic agents are provided.

Provided herein are also articles of manufacture that include a compound of the present disclosure or a pharmaceutically acceptable salt, tautomer, stereoisomer, mixture of stereoisomers, prodrug, or deuterated analog thereof in a suitable container. The container may be a vial, jar, ampoule, preloaded syringe, implant, or intravenous bag.

In some embodiments, the present disclosure relates to a kit comprising a compound of formula I, II, III, IV, V, VI, VII, VIIa, VIIb, VIII, or IX, or a pharmaceutically acceptable salt thereof. In one embodiment, the kit may comprise one, two, three, or four additional therapeutic agents as described hereinbefore. The kit may further comprise instructions for use, e.g., for use in inhibiting an HIV integrase, such as for use in treating an HIV infection or AIDS, or as a research tool. The instructions for use are generally written instructions, although electronic storage media (e.g., magnetic diskette or optical disk) containing instructions are also acceptable.

In some embodiments, the present disclosure also relates to a pharmaceutical kit comprising one or more containers comprising a compound formula I, II, III, IV, V, VI, VII, VIIa, VIIb, VIII, or IX, or a pharmaceutically acceptable salt thereof. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

In some embodiments, disclosed herein are articles of manufacture comprising a unit dosage of a compound of formula I, II, III, IV, V, VI, VII, VIIa, VIIb, VIII, or IX, or a pharmaceutically acceptable salt thereof, in suitable packaging for use in the methods described herein. Suitable packaging is known in the art and includes, for example, vials, vessels, ampules, bottles, jars, flexible packaging and the like. An article of manufacture may further be sterilized and/or sealed.

### Abbreviations

ACN acetonitrile
Ac₂O acetic anhydride
AcOH acetic acid
AUC area under the curve
AZT azidothymidine (zidovudine)
BLQ below the limit of quantitation
Bn benzyl
BSA N,O-bis(trimethylsilyl)acetamide
bSA bovine serum albumin
CC₅₀ half maximal cytotoxic concentration
Cmax maximum serum concentration
d doublet
DCM dichloromethane
dd doublet of doublets
dCTP deoxycytidine triphosphate
ddd doublet of doublet of doublets
dGTP Deoxyguanosine triphosphate
DMF N,N-dimethylformamide
DMSO dimethyl sulfoxide
dNTP deoxynucleoside triphosphate
dq doublet of quartets
dt doublet of triplets
DTT dithiothreitol
EC₅₀ half maximal effective concentration
EDTA ethylenediamine tetraacetic acid
eq equivalents
EtOAc ethyl acetate
EtOH ethanol
FBS fetal bovine serum
g gram(s)
h or hr hour(s)
HIV human immunodeficiency virus
HPLC high performance liquid chromatography
Hz hertz
IC₅₀ half maximal inhibitory concentration
J coupling constant
LCMS or LC-MS liquid chromatography-mass spectrometry
M molar
m multiplet
MeOH methanol
mg milligram(s)
MHz megahertz
min minute(s)
mL or ml milliliter(s)
mm millimeter(s)
mmol millimole(s)
ms molecular sieves
MS mass spectrometry
MT-4 or MT4 metallothionein 4 human T cell line
NaOEt sodium ethoxide
NIS N-iodosuccinimide
NMR nuclear magnetic resonance
PBMC peripheral blood mononuclear cell
PEG polyethylene glycol
prep preparative
P/S penicillin-streptomycin
q quartet
RIPA Radioimmunoprecipitation assay
RPMI Roswell Park Memorial Institute (culture medium)
RT reverse transcriptase
s singlet
SD standard deviation
SNI single nucleotide incorporation
SNIR single nucleotide incorporation rate
t triplet
t_{1/2} half-life
td triplet of doublets
TBAF tetrabutylammonium fluoride
TFV tenofovir
THF tetrahydrofuran
TLC thin layer chromatography
TPGS tocopheryl polyethylene glycol succinate
Tris tris(hydroxymethyl)aminomethane
μ micron
μL microliter
μM micromolar
v/v volume per volume
xg times gravity

The following examples are provided for purposes of illustration, not limitation.

### EXAMPLES

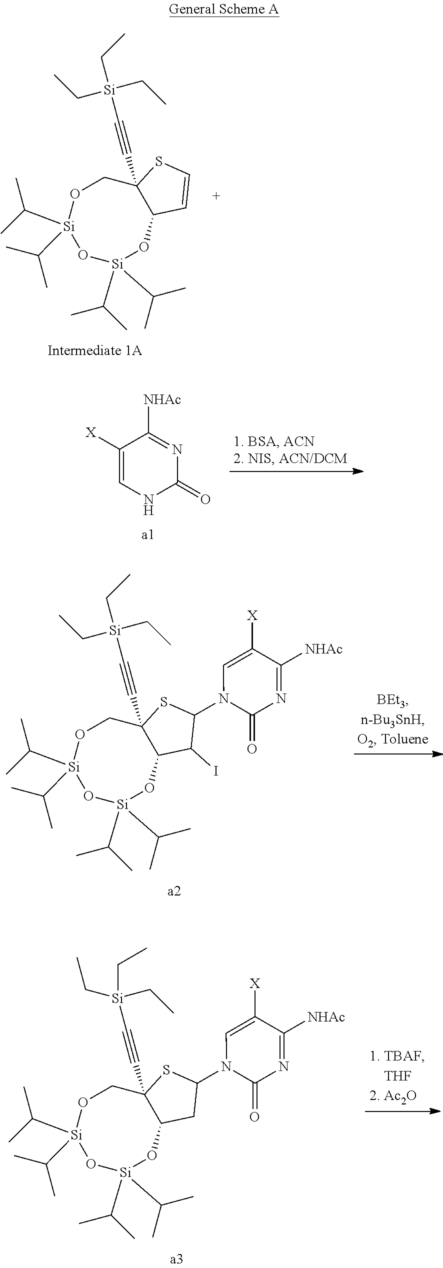

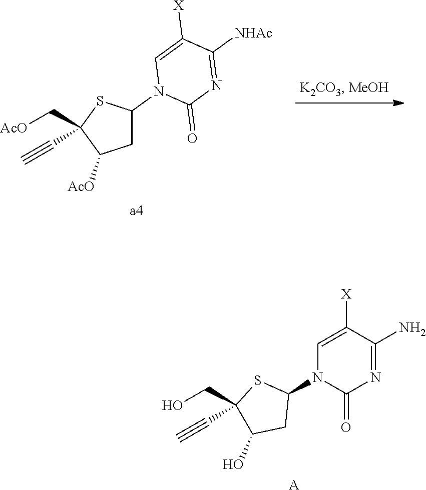

General Scheme A shows a general synthesis of compounds represented by formula A starting with the silylation of a1 with a silylating reagent (e.g., BSA). Addition of this silylated base to Intermediate 1A (ACS Med. Chem. Lett. 2011, 2, pp. 692-697) is accomplished through activation of the olefin with an iodine source (e.g., NIS), furnishing a2. Removal of the iodine with a reducing reagent (e.g., n-BusSnH) yields a3. Removal of the silyl protecting groups with a fluoride source (e.g., TBAF) followed by per-acylation with an acylating reagent (e.g., acetic anhydride) to facilitate purification provides a4. Removal of the acyl groups of a4 under basic solvolysis conditions (e.g., K₂CO₃, MeOH) yields compounds represented by formula A.

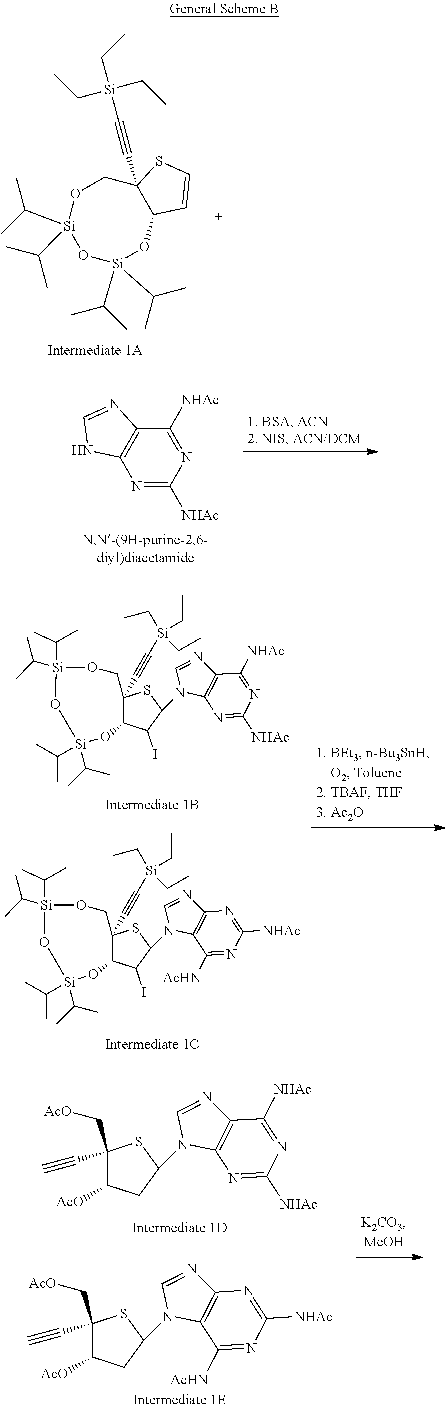 [SMILES: CC[Si](C#C[C@@]12CO[Si](C(C)C)(C(C)C)O[Si](C(C)C)(C(C)C)O[C@H]1C=CS2)(CC)CC ; CC(=O)Nc1nc(NC(C)=O)c2nc[nH]c2n1 ; CC[Si](C#C[C@@]12CO[Si](C(C)C)(C(C)C)O[Si](C(C)C)(C(C)C)O[C@H]1C(I)C(n1cnc3c(NC(C)=O)nc(NC(C)=O)nc31)S2)(CC)CC ; CC[Si](C#C[C@@]12CO[Si](C(C)C)(C(C)C)O[Si](C(C)C)(C(C)C)O[C@H]1C(I)C(n1cnc3nc(NC(C)=O)nc(NC(C)=O)c31)S2)(CC)CC ; C#C[C@]1(COC(C)=O)SC(n2cnc3c(NC(C)=O)nc(NC(C)=O)nc32)C[C@@H]1OC(C)=O ; C#C[C@]1(COC(C)=O)SC(n2cnc3nc(NC(C)=O)nc(NC(C)=O)c32)C[C@@H]1OC(C)=O]

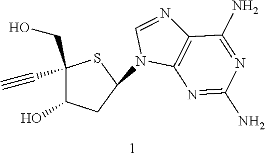 [SMILES: C#C[C@]1(CO)S[C@@H](n2cnc3c(N)nc(N)nc32)C[C@@H]1O]

General Scheme B shows a general synthesis of compound 1 starting with the silylation of N,N′-(9H-purine-2,6-diyl)diacetamide with a silylating reagent (e.g., BSA). Addition of this silylated base to Intermediate 1A is accomplished through activation of the olefin with an iodine source (e.g., NIS), furnishing a mixture Intermediates 1B and 1C. Removal of the iodine is accomplished with a reducing reagent (e.g., n-Bu₃SnH). Removal of the silyl protecting groups is accomplished with a fluoride source (e.g., TBAF) and the resulting mixture is per-acylated with an acylating reagent (e.g., acetic anhydride) to provide a mixture of Intermediates 1D and 1E. Removal of the acyl groups of the mixture of Intermediate 1D and 1E under basic solvolysis conditions (e.g., K₂CO₃, MeOH) yields compound 1 after separation from the other isomers.

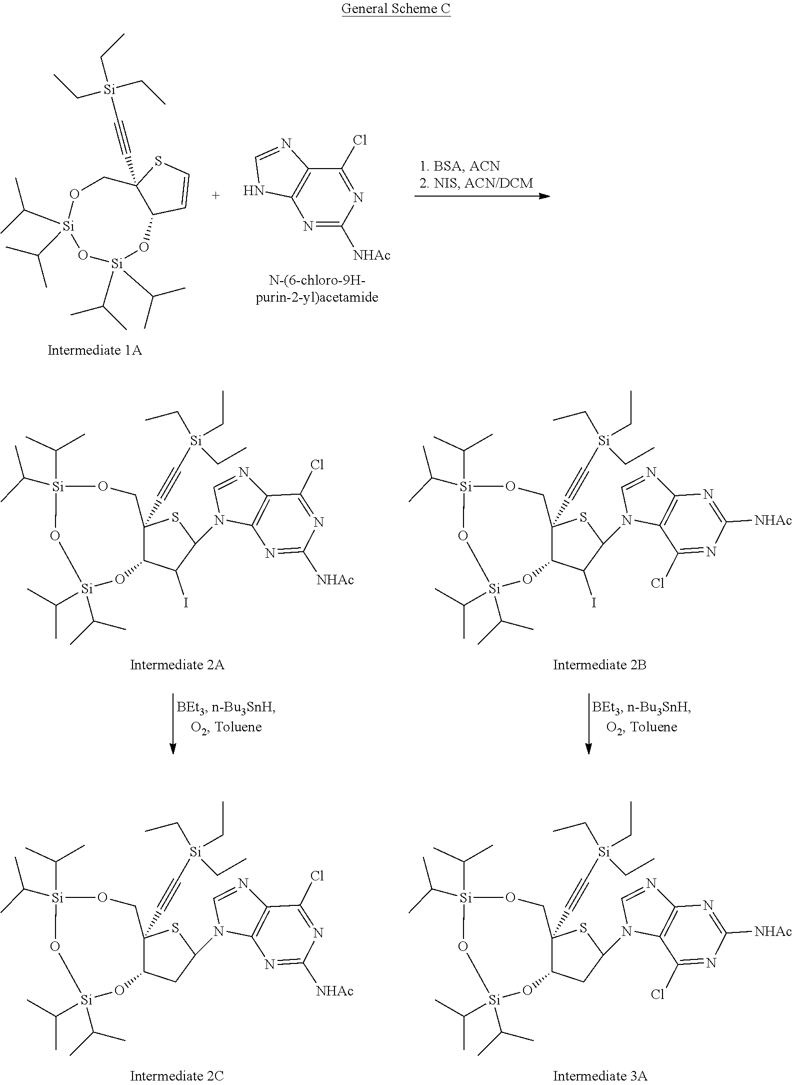

General Scheme C shows a general synthesis of Intermediates 2C and 3A starting with the silylation of N-(6-chloro-9H-purine-2-yl)acetamide with a silylating reagent (e.g., BSA). Addition of this silylated base to Intermediate 1A is accomplished through activation of the olefin with an iodine source (e.g., NIS), furnishing a mixture of Intermediates 2A and 2B that can be separated by chromatography. Removal of the iodine of Intermediate 2A is accomplished with a reducing reagent (e.g., n-Bu₃SnH) to yield Intermediate 2C. Intermediate 2B is converted to Intermediate 3A in a similar fashion as the conversion of Intermediate 2A to 2C.

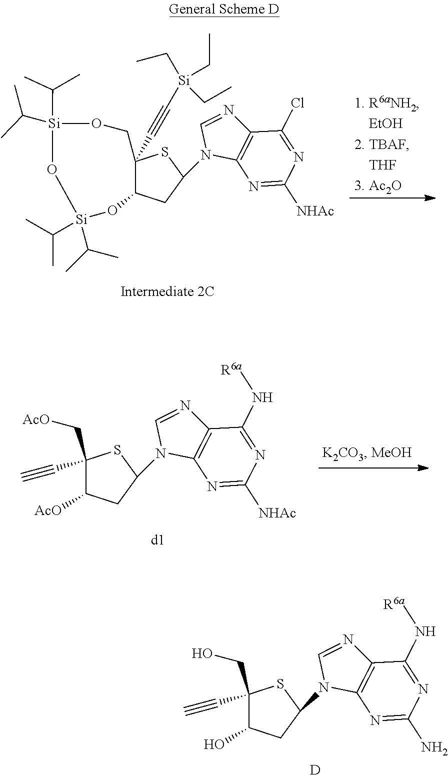

General Scheme D shows a general synthesis of compounds represented by formula D. Installation of the R^{6a}NH— present in d1 is accomplished by a nucleophilic aryl substitution reaction, replacing the Cl in Intermediate 2C with R^{6a}NH₂ (e.g., cPr-NH₂). Removal of the silyl protecting groups is accomplished with a fluoride source (e.g., TBAF) and the resulting mixture is per-acylated with an acylating reagent (e.g., acetic anhydride) to provide d1. Removal of the acyl groups of d1 under basic solvolysis conditions (e.g., K₂CO₃, MeOH) yields compounds represented by formula D after separation from the other isomer.

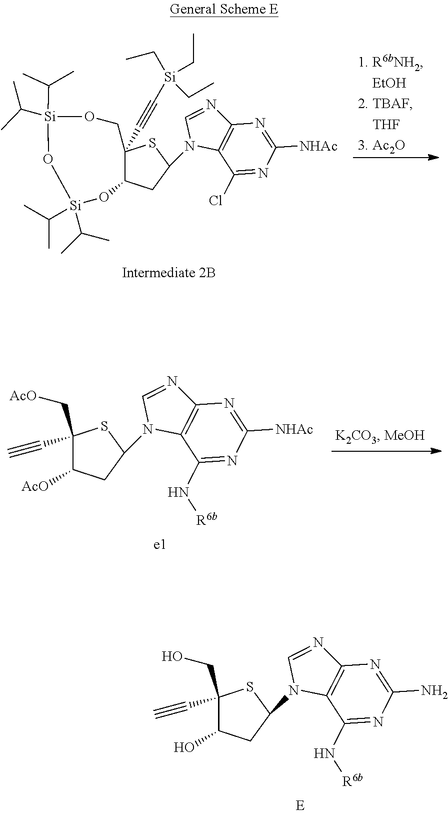

General Scheme E shows a general synthesis of compounds represented by formula E. This is accomplished in a similar manner as is described in General Scheme D.

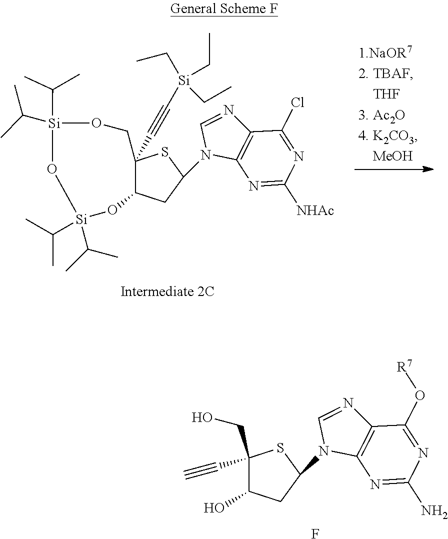

General Scheme F shows a general synthesis of compounds represented by formula F. Installation of the R⁷O— present in F is accomplished by a nucleophilic aryl substitution reaction, replacing the C₁ in Intermediate 2C with a sodium alkoxide (e.g., NaOEt) or other alkoxide source. Removal of the silyl protecting groups is accomplished with a fluoride source (e.g., TBAF), the resulting mixture is per-acylated with an acylating reagent (e.g., acetic anhydride) to facilitate purification and the acetates are subsequently removed under basic solvolysis conditions (e.g., K₂CO₃, MeOH) to yield compounds represented by formula F after separation from the other isomer.

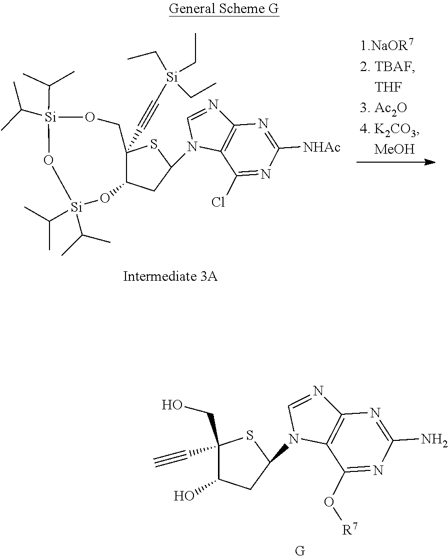

General Scheme G shows a general synthesis of compounds represented by formula G. This is accomplished in a similar manner as is described in General Scheme F.

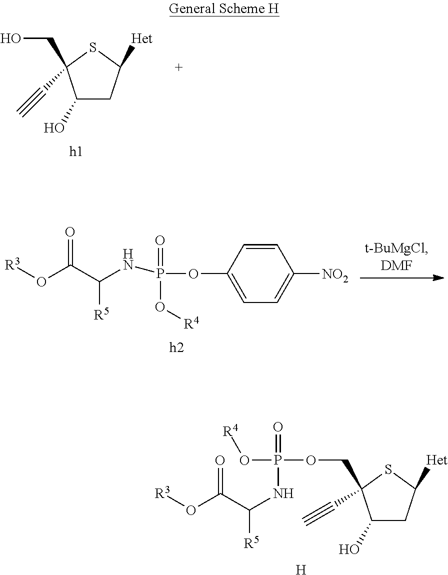

General Scheme H shows a general synthesis of compounds with the formula represented by H. Compounds with the formula represented by hi are deprotonated by tBuMgCl in a solution of DMF. This mixture is then treated with a solution of compounds with formula h2 in DMF to yield compounds with formula H.

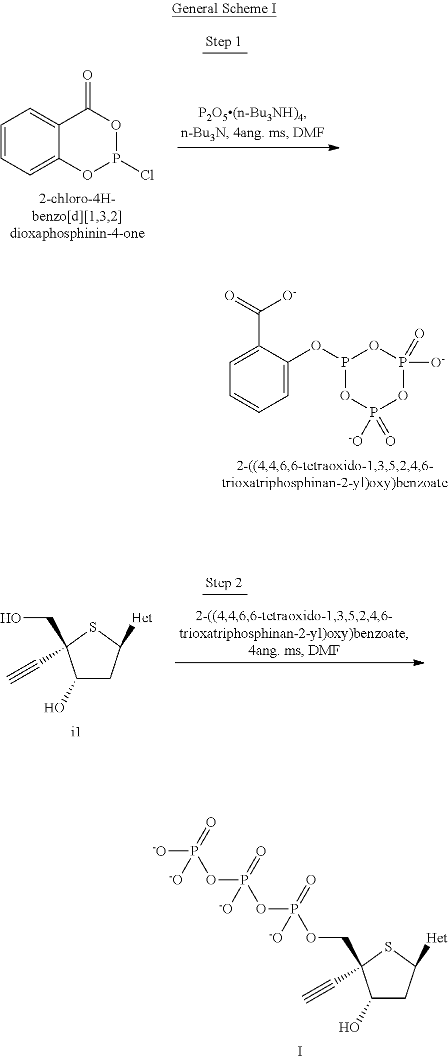

General Scheme I shows a general synthesis of compounds with formula I. A mixture of tributylammonium pyrophosphate, a solvent (e.g. DMF) and a trialkyl amine base (e.g. tributylamine) are added to a mixture of 2-chloro-4H-benzo[d][1,3,2]dioxaphosphinin-4-one, molecular sieves (e.g. 4 angstrom molecular sieves) and a solvent (e.g. DMF) to generate a solution of 2-((4,4,6,6-tetraoxido-1,3,5,2,4,6-trioxatriphosphinan-2-yl)oxy)benzoate. This solution is then added to a mixture of a compounds of formula i1 and molecular sieves (e.g., 4 angstrom molecular sieves) to yield compounds with formula I.

### Example 1: ((2R,3S,5R)-5-(2,6-Diamino-9H-purin-9-yl)-2-ethynyl-2-(hydroxymethyl)tetrahydrothiophen-3-ol) (1)

### Synthesis of Mixture of Intermediates 1B (N,N′-(9-((6aR,9aR)-9-iodo-2,2,4,4-tetraisopropyl-6a-((triethylsilyl)ethynyl)tetrahydro-6H-thieno[3,2-f][1,3,5,2,4]trioxadisilocin-8-yl)-9H-purine-2,6-diyl)diacetamide) and 1C (N,N′-(7-((6aR,9aR)-9-iodo-2,2,4,4-tetraisopropyl-6a-((triethylsilyl)ethynyl)tetrahydro-6H-thieno[3,2-f][1,3,5,2,4]trioxadisilocin-8-yl)-7H-purine-2,6-diyl)diacetamide)

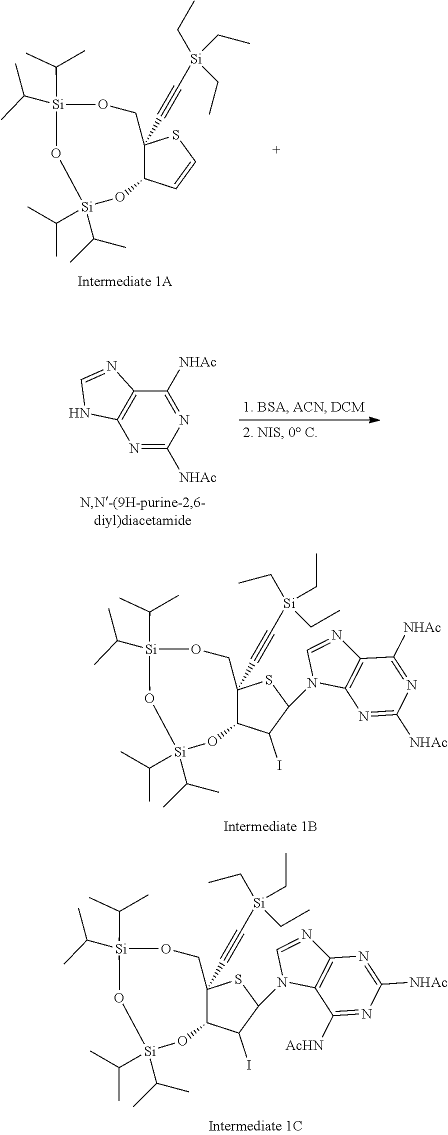

N,O-Bis(trimethylsilyl)acetamide (357 μL, 1.46 mmol) was added to a suspension of N,N′-(9H-purine-2,6-diyl)diacetamide (171 mg, 0.731 mmol) in acetonitrile (4 mL) and dichloromethane (5 mL) and heated at 70° C. After 2 h the mixture was still a suspension. Additional N,O-Bis(trimethylsilyl)acetamide (357 μL, 1.46 mmol) was added. After another 3 h the reaction mixture was cooled to 0° C. and a solution of intermediate 1A (250 mg, 0.487 mmol) and N-Iodosuccinimide (164 mg, 0.731 mmol) in acetonitrile (4 mL) was added. After 3 days at 0° C. the reaction was allowed to warm to room temperature. The mixture was diluted with dichloromethane (50 mL) and aqueous sodium thiosulfate (1M, 5 mL) was added. The aqueous phase was extracted with dichloromethane (10 mL). The combined organic phases were dried over sodium sulfate and the solvent was removed under reduced pressure. The residue was subjected to flash chromatography (0-100% ethyl acetate/hexanes). The fractions containing product were combined and the solvent was removed under reduced pressure to provide the mixture of intermediates 1B and 1C: LCMS: MS m/z=872.835 [M+1]. LCMS: MS m/z 872.949 [M+1].

### Synthesis of Mixture of Intermediates 1D (((2R,3S)-3-acetoxy-5-(2,6-diacetamido-9H-purin-9-yl)-2-ethynyltetrahydrothiophen-2-yl)methyl acetate) and 1E (((2R,3S)-3-acetoxy-5-(2,6-diacetamido-7H-purin-7-yl)-2-ethynyltetrahydrothiophen-2-yl)methyl acetate)

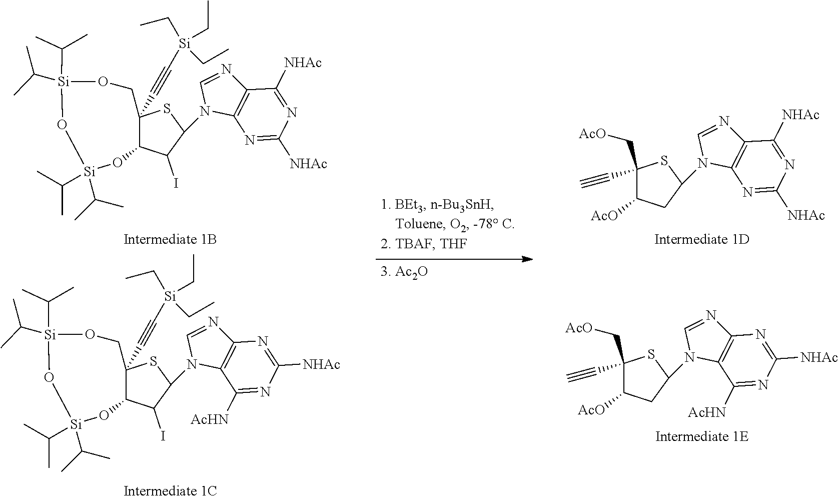 [SMILES: CC[Si](C#C[C@@]12CO[Si](C(C)C)(C(C)C)O[Si](C(C)C)(C(C)C)O[C@H]1C(I)C(n1cnc3c(NC(C)=O)nc(NC(C)=O)nc31)S2)(CC)CC ; C#C[C@]1(COC(C)=O)SC(n2cnc3c(NC(C)=O)nc(NC(C)=O)nc32)C[C@@H]1OC(C)=O ; CC[Si](C#C[C@@]12CO[Si](C(C)C)(C(C)C)O[Si](C(C)C)(C(C)C)O[C@H]1C(I)C(n1cnc3nc(NC(C)=O)nc(NC(C)=O)c31)S2)(CC)CC ; C#C[C@]1(COC(C)=O)SC(n2cnc3nc(NC(C)=O)nc(NC(C)=O)c32)C[C@@H]1OC(C)=O]

Tri-n-butyltin hydride (76.3 mg, 0.263 mmol) was added to triethylborane (1 M, 0.0346 mL, 0.0346 mmol) and a mixture of Intermediates 1B and 1C (152 mg, 0.173 mmol) in toluene (10 mL), under oxygen at −78° C. After 2 h the reaction mixture was quenched with methanol (2 mL) and 1 drop of water at −78° C. The reaction mixture was warmed, the solvent was removed under reduced pressure and the residue was azeotroped with toluene (3×10 mL). The residue was placed under high vacuum for 16 h. The resulting mixture was carried forward into the next reaction.

The mixture was taken up in THF (5 mL) and tetrabutylammonium fluoride solution (1M, 0.87 mL, 0.870 mmol) was added. After 20 min acetic anhydride (82 μL, 0.870 mmol) was added. After 2 h the reaction was quenched with saturated sodium bicarbonate (20 mL). The aqueous phase was extracted with dichloromethane (3×20 mL). The combined organic phases were dried over sodium sulfate and the solvent was removed under reduced pressure. The residue was subjected to flash chromatography (0-10% methanol/dichoromethane). The fractions containing product were combined and the solvent was removed under reduced pressure, providing a mixture of intermediates 1D and 1E: LCMS: MS m/z=474.752 [M+1]. LCMS: MS m/z=473.997 [M+1].

### Synthesis of ((2R,3S,5R)-5-(2,6-diamino-9H-purin-9-yl)-2-ethynyl-2-(hydroxymethyl)tetrahydrothiophen-3-ol) (1)

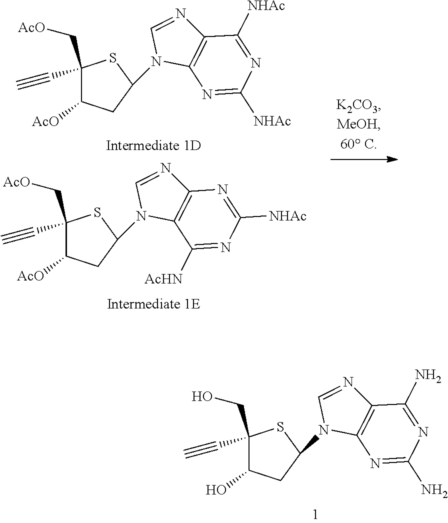 [SMILES: C#C[C@]1(COC(C)=O)SC(n2cnc3c(NC(C)=O)nc(NC(C)=O)nc32)C[C@@H]1OC(C)=O ; C#C[C@]1(COC(C)=O)SC(n2cnc3nc(NC(C)=O)nc(NC(C)=O)c32)C[C@@H]1OC(C)=O ; C#C[C@]1(CO)S[C@@H](n2cnc3c(N)nc(N)nc32)C[C@@H]1O]

A mixture of intermediates 1D and 1E (30 mg, 0.0632 mmol), potassium carbonate (43.7 mg, 0.316 mmol) in methanol (2 mL) was stirred at room temperature for 18 h. The mixture was heated at 50° C. for 30 min. More potassium carbonate (87.4 mg, 0.632 mmol) was added and the reaction mixture was heated at 60° C. After another hour the reaction mixture was cooled to room temperature and neutralized with acetic acid (57.0 mg, 0.948 mmol). Water (1 mL) was added and the mixture was subjected to prep HPLC. The fractions containing product were combined and subjected to lyophilization, providing compound 1. ¹H NMR (400 MHz, DMSO-d₆) δ 8.05 (s, 1H), 6.71 (s, 2H), 6.07 (t, J=6.0 Hz, 1H), 5.83 (s, 2H), 5.60-5.58 (m, 2H), 4.47 (dd, J=7.5, 4.4 Hz, 1H), 3.79 (d, J=11.3 Hz, 1H), 3.69 (d, J=11.4 Hz, 1H), 3.37 (s, 1H), 2.68-2.62 (m, 1H), 2.54-2.48 (m, 1H). LCMS: MS m/z=306.965 [M+1].

### Example 2: ((2R,3S,5R)-5-(2-Amino-6-(cyclopropylamino)-9H-purin-9-yl)-2-ethynyl-2-(hydroxymethyl)tetrahydrothiophen-3-ol) (2)

### Synthesis of Intermediate 2A (N-(6-chloro-9-((6aR,9aR)-9-iodo-2,2,4,4-tetraisopropyl-6a-((triethylsilyl)ethynyl)tetrahydro-6H-thieno[3,2-f][1,3,5,2,4]trioxadisilocin-8-yl)-9H-purin-2-yl)acetamide) and Intermediate 2B (N-(6-chloro-7-((6aR,9aR)-9-iodo-2,2,4,4-tetraisopropyl-6a-((triethylsilyl)ethynyl)tetrahydro-6H-thieno[3,2-f][1,3,5,2,4]trioxadisilocin-8-yl)-7H-purin-2-yl)acetamide)

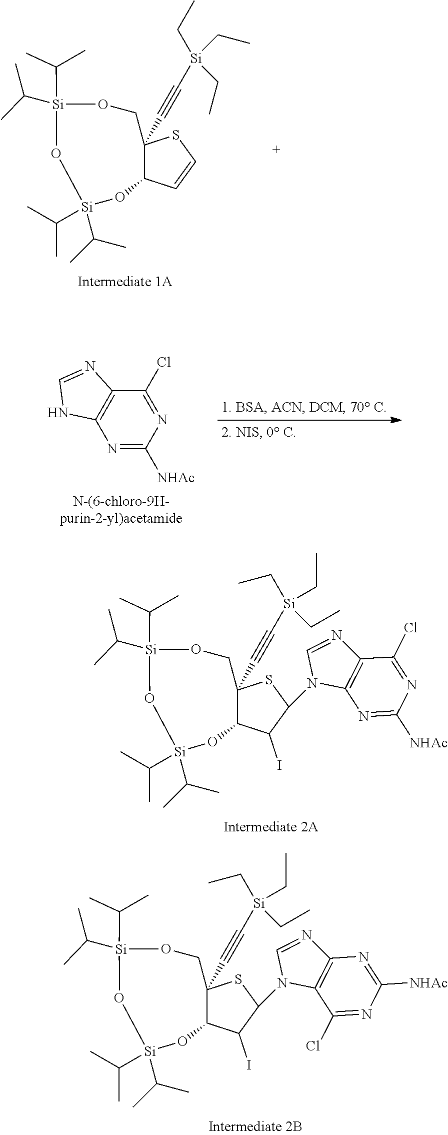

N,O-Bis(trimethylsilyl)acetamide (572 μL, 2.34 mmol) was added to a suspension of N-(6-chloro-9H-purin-2-yl)acetamide (248 mg, 1.17 mmol) in acetonitrile (10 mL) in an oven dried flask under argon. After 1 h the solution was cooled to 0° C. under Ar. A solution of intermediate 1A (ACS Med. Chem. Lett. 2011, 2, pp. 692-697; 400 mg, 0.780 mmol) and N-iodosuccinimide (263 mg, 1.17 mmol) in dichloromethane (10 mL) was added. After 18 h at 0° C. a solution resulted (no solid). The reaction was diluted with ethyl acetate (20 mL) and washed with aqueous sodium thiosufate (1M, 10 mL) and brine (10 mL). The organic phase was dried over sodium sulfate and the solvent was removed under reduced pressure. The residue was subjected to flash chromatography (0-100% ethyl acetate/dichloromethane). The fractions containing the products were combined and solvent was removed under reduced pressure to provide, separately, intermediates 2A and 2B.

Intermediate 2A (N-(6-chloro-9-((6aR,9aR)-9-iodo-2,2,4,4-tetraisopropyl-6a-((triethylsilyl)ethynyl)tetrahydro-6H-thieno[3,2-f][1,3,5,2,4]trioxadisilocin-8-yl)-9H-purin-2-yl)acetamide): ¹H NMR (400 MHz, Chloroform-d) δ 8.48 (s, 1H), 8.01 (s, 1H), 6.53 (d, J=3.0 Hz, 1H), 4.83 (dd, J=6.9, 3.1 Hz, 1H), 4.10 (d, J=12.5 Hz, 1H), 4.04 (d, J=12.5 Hz, 1H), 3.97 (d, J=6.9 Hz, 1H), 2.71 (s, 3H), 1.17-1.09 (m, 24H), 1.06 (t, J=7.9 Hz, 9H), 1.01-0.97 (m, 4H), 0.68 (q, J=7.9 Hz, 6H). LCMS: MS m/z=849.898 [M+1].

Intermediate 2B (N-(6-chloro-7-((6aR,9aR)-9-iodo-2,2,4,4-tetraisopropyl-6a-((triethylsilyl)ethynyl)tetrahydro-6H-thieno[3,2-f][1,3,5,2,4]trioxadisilocin-8-yl)-7H-purin-2-yl)acetamide): ¹H NMR (400 MHz, Chloroform-d) δ 9.11 (s, 1H), 8.00 (s, 1H), 6.74 (d, J=2.1 Hz, 1H), 4.61 (d, J=6.0 Hz, 1H), 4.11 (s, 2H), 3.94 (d, J=6.0 Hz, 1H), 2.65 (s, 3H), 1.22-1.10 (m, 24H), 1.06 (t, J=7.9 Hz, 9H), 0.94-0.90 (m, 4H), 0.72-0.64 (m, 6H). LCMS: MS m/z=849.918 [M+1].

### Synthesis of Intermediate 2C (N-(6-chloro-9-((6aR,9aS)-2,2,4,4-tetraisopropyl-6a-((triethylsilyl)ethynyl)tetrahydro-6H-thieno[3,2-f][1,3,5,2,4]trioxadisilocin-8-yl)-9H-purin-2-yl)acetamide)

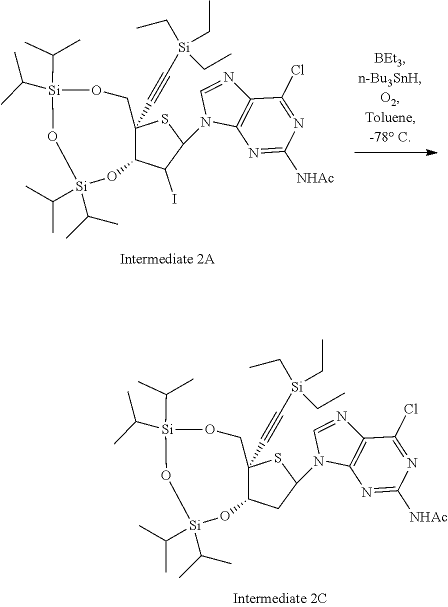 [SMILES: CC[Si](C#C[C@@]12CO[Si](C(C)C)(C(C)C)O[Si](C(C)C)(C(C)C)O[C@H]1C(I)C(n1cnc3c(Cl)nc(NC(C)=O)nc31)S2)(CC)CC ; CC[Si](C#C[C@@]12CO[Si](C(C)C)(C(C)C)O[Si](C(C)C)(C(C)C)O[C@H]1CC(n1cnc3c(Cl)nc(NC(C)=O)nc31)S2)(CC)CC]

Tri-n-butyltin hydride (46.9 μL, 0.175 mmol) was added to triethylborane (1 M, 23.3 μL, 0.0233 mmol) and intermediate 2A (99 mg, 0.116 mmol) in toluene (5 mL), under oxygen at −78° C. After 2 h more triethylborane (1 M, 116.5 μL, 0.116 mmol) and tri-n-butyltin hydride (46.9 μL, 0.175 mmol) were added. After 4 h the reaction mixture was quenched with methanol (5 mL) and 1 drop of water at −78° C. The reaction mixture was warmed to room temperature and the solvent was removed under reduced pressure. The residue was azeotroped with toluene (10 mL). The residue was subjected to silica gel column chromatography (0-100% ethyl acetate/dichloromethane). The fractions containing product were combined and the solvent was removed under reduced pressure, providing a mixture containing intermediate 2C. LCMS: MS m/z=724.099 [M+1].

### Synthesis of Intermediate 2D ((2R,3S)-5-(2-acetamido-6-(cyclopropylamino)-9H-purin-9-yl)-2-(acetoxymethyl)-2-ethynyltetrahydrothiophen-3-yl acetate)

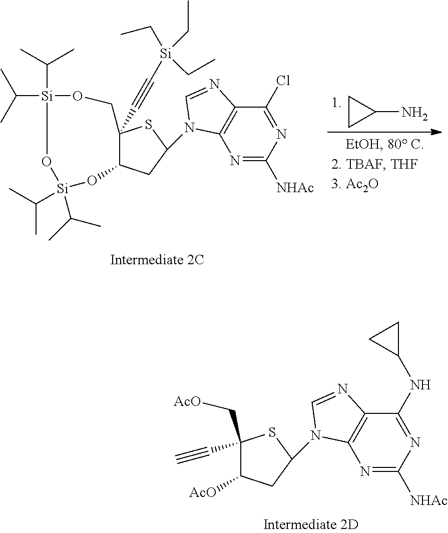

A solution of cyclopropylamine (0.416 mL, 6.00 mmol), and a mixture containing intermediate 2C (86.9 mg) and ethanol (3 mL) was heated at 80° C. for 45 minutes. The solvent was removed under reduced pressure and the residue was co-evaporated with toluene (5 mL). The residue was taken up in tetrahydrofuran (3 mL) and tetrabutylammonium fluoride (1 M, 0.600 mL, 0.600 mmol) was added. Acetic anhydride (0.6 mL, 0.600 mmol) was added. After 30 minutes the reaction mixture was quenched with water (0.5 mL) and saturated sodium bicarbonate (3 mL). The mixture was extracted with ethyl acetate (3×10 mL). The organic phase was dried over sodium sulfate and the solvent was removed under reduced pressure. The residue was subjected to flash chromatography (0-30% methanol/dichloromethane). The fractions containing product were combined and the solvent was removed under reduced pressure, providing a mixture containing intermediate 2D, which was carried forward to the next step without further purification. LCMS: MS m/z=473.015 [M+1].

### Synthesis of ((2R,3S,5R)-5-(2-amino-6-(cyclopropylamino)-9H-purin-9-yl)-2-ethynyl-2-(hydroxymethyl)tetrahydrothiophen-3-ol) (2)

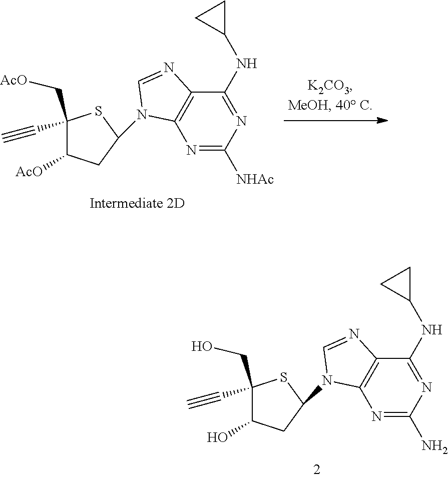 [SMILES: C#C[C@]1(COC(C)=O)SC(n2cnc3c(NC4CC4)nc(NC(C)=O)nc32)C[C@@H]1OC(C)=O ; C#C[C@]1(CO)S[C@@H](n2cnc3c(NC4CC4)nc(N)nc32)C[C@@H]1O]

A mixture containing intermediate 2D (47.3 mg), potassium carbonate (137 mg, 0.995 mmol) and methanol (4 mL) was stirred at 40° C. for 1 hour. More potassium carbonate (137 mg, 0.995 mmol) was added and the mixture was stirred at 60° C. for 3 hours. The reaction mixture was cooled and neutralized with acetic acid (0.2 mL, 0.2 mmol). The solution was subjected to preparative HPLC (0-40% acetonitrile/water (no acid additive in mobile phase)). The fractions containing product were combined and subjected to lyophilization, providing compound 2. ¹H NMR (400 MHz, DMSO-d₆) δ 8.04 (s, 1H), 7.42-7.26 (m, 1H), 6.08 (dd, J=6.6, 5.3 Hz, 1H), 5.91 (s, 2H), 5.68 (s, 1H), 4.47 (dd, J=7.5, 4.4 Hz, 1H), 3.78 (d, J=11.4 Hz, 1H), 3.69 (d, J=11.4 Hz, 1H), 3.37 (s, 1H), 3.02 (s, 1H), 2.70-2.51 (m, 3H), 0.70-0.61 (m, 2H), 0.61-0.54 (m, 2H). LCMS: MS m/z=346.999 [M+1].

### Example 3: ((2R,3S,5R)-5-(2-Amino-6-(cyclopropylamino)-7H-purin-7-yl)-2-ethynyl-2-(hydroxymethyl)tetrahydrothiophen-3-ol) (3)

### Synthesis of Intermediate 3A (N-(6-chloro-7-((6aR,9aS)-2,2,4,4-tetraisopropyl-6a-((triethylsilyl)ethynyl)tetrahydro-6H-thieno[3,2-f][1,3,5,2,4]trioxadisilocin-8-yl)-7H-purin-2-yl)acetamide)

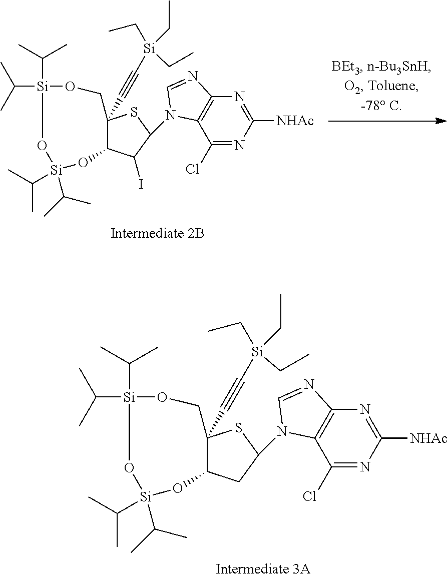 [SMILES: CC[Si](C#C[C@@]12CO[Si](C(C)C)(C(C)C)O[Si](C(C)C)(C(C)C)O[C@H]1C(I)C(n1cnc3nc(NC(C)=O)nc(Cl)c31)S2)(CC)CC ; CC[Si](C#C[C@@]12CO[Si](C(C)C)(C(C)C)O[Si](C(C)C)(C(C)C)O[C@H]1CC(n1cnc3nc(NC(C)=O)nc(Cl)c31)S2)(CC)CC]

Tri-n-butyltin hydride (120 μL, 0.455 mmol) was added to triethylborane (1 M, 60.7 L, 0.0607 mmol) and intermediate 2B (258 mg, 0.303 mmol) in toluene (5 mL), under oxygen at −78° C. After 2 hours more triethylborane (1 M, 300 μL, 0.300 mmol) and tri-n-butyltin hydride (120 μL, 0.455 mmol) were added. After 4 h hours the reaction mixture was quenched with methanol (5 mL) and 1 drop of water at −78° C. The reaction mixture was warmed to room temperature and the solvent was removed under reduced pressure. The residue was azeotroped with toluene (10 mL). The residue was subjected to flash chromatography (0-100% ethyl acetate/dichloromethane). The fractions containing product were combined and the solvent was removed under reduced pressure, providing a mixture containing 3A, which was carried through to the next step. LCMS: MS m/z=724.087 [M+1].

### Synthesis of Intermediate 3B ((2R,3S)-5-(2-acetamido-6-(cyclopropylamino)-7H-purin-7-yl)-2-(acetoxymethyl)-2-ethynyltetrahydrothiophen-3-yl acetate)

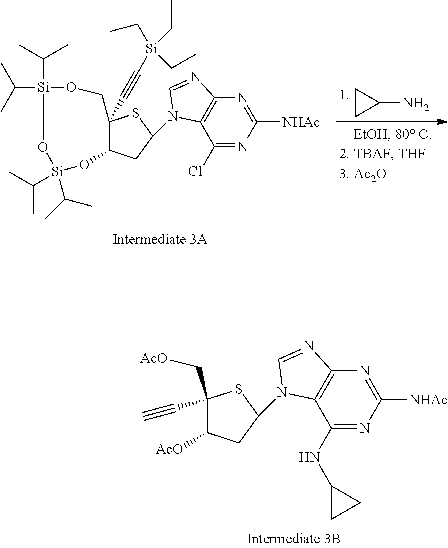

A solution of cyclopropylamine (0.478 mL, 6.90 mmol), a mixture containing intermediate 3A (100 mg, 0.138 mmol) and ethanol (3 mL) was heated at 80° C. for 90 minutes. The solvent was removed under reduced pressure and the residue was co-evaporated with toluene (5 mL). The residue was taken up in tetrahydrofuran (3 mL) and tetrabutylammonium fluoride (1 M, 0.690 mL, 0.690 mmol) was added. Acetic anhydride (0.84 mL, 0.828 mmol) was added. After 1 hour the reaction mixture was quenched with water (0.5 mL) and saturated sodium bicarbonate (3 mL). The mixture was extracted with ethyl acetate (3×10 mL). The organic phase was dried over sodium sulfate and the solvent was removed under reduced pressure. The residue was subjected to flash chromatography (0-30% methanol/dichloromethane). The fractions containing product were combined and the solvent was removed under reduced pressure, providing intermediate 3B. ¹H NMR (400 MHz, Chloroform-d) δ 8.27 (s, 1H), 6.39 (s, 1H), 5.53 (s, 1H), 4.52 (d, J=11.7 Hz, 1H), 4.35 (d, J=11.7 Hz, 1H), 3.04-2.92 (m, 1H), 2.86 (s, 2H), 2.74 (s, 1H), 2.69 (s, 3H), 2.26-2.19 (m, 3H), 2.18 (d, J=0.7 Hz, 3H), 1.47-1.27 (m, 1H), 0.95 (t, J=7.1 Hz, 2H), 0.76 (d, J=21.4 Hz, 2H). LCMS: MS m/z=472.936 [M+1].

### Synthesis of ((2R,3S,5R)-5-(2-amino-6-(cyclopropylamino)-7H-purin-7-yl)-2-ethynyl-2-(hydroxymethyl)tetrahydrothiophen-3-ol) (3)

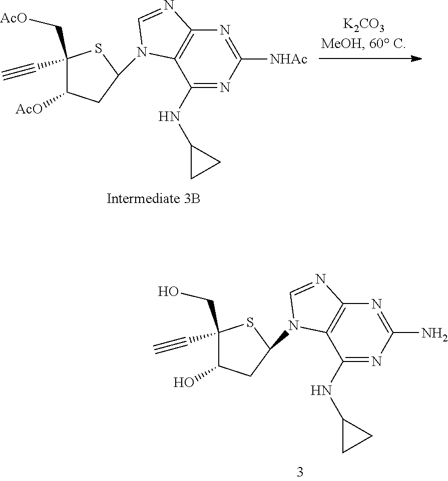 [SMILES: C#C[C@]1(COC(C)=O)SC(n2cnc3nc(NC(C)=O)nc(NC4CC4)c32)C[C@@H]1OC(C)=O ; C#C[C@]1(CO)S[C@@H](n2cnc3nc(N)nc(NC4CC4)c32)C[C@@H]1O]

A mixture of intermediate 3B (43.1 mg, 0.092 mmol), potassium carbonate (126 mg, 0.912 mmol) and methanol (4 mL) was stirred at 40° C. for 1 hour. More potassium carbonate (126 mg, 0.912 mmol) was added and the mixture was stirred at 60° C. for 3 hours. The reaction mixture was cooled and neutralized with acetic acid (0.25 mL, 0.25 mmol). The solution was subjected to preparative HPLC (0-40% acetonitrile/water neutral). The fractions containing product were combined and subjected to lyophilization, providing compound 3. ¹H NMR (400 MHz, DMSO-d₆) δ 8.46 (s, 1H), 6.57 (d, J=3.1 Hz, 1H), 6.34 (dd, J=6.3, 2.0 Hz, 1H), 5.67 (s, 2H), 5.57 (s, 1H), 4.27-4.14 (m, 1H), 3.68 (d, J=3.7 Hz, 2H), 3.45 (s, 1H), 3.17, (m, 1H), 2.95-2.82 (m, 1H), 2.58 (ddd, J=13.0, 11.1, 6.3 Hz, 1H), 2.24 (ddd, J=13.1, 4.8, 2.0 Hz, 1H), 0.74-0.61 (m, 2H), 0.66-0.49 (m, 2H). LCMS: MS m/z=346.993 [M+1].

### Example 4: ((2R,3S,5R)-5-(2-Amino-6-ethoxy-9H-purin-9-yl)-2-ethynyl-2-(hydroxymethyl)tetrahydrothiophen-3-ol) (4)

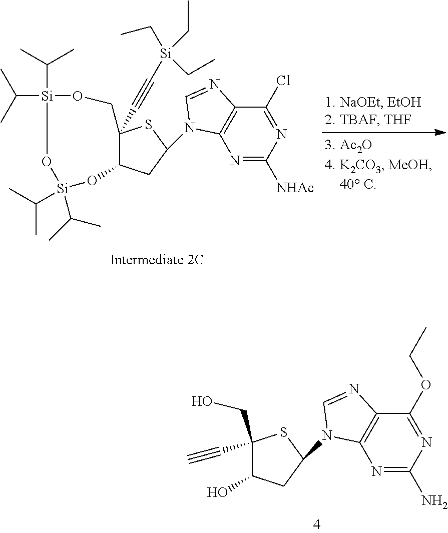 [SMILES: CC[Si](C#C[C@@]12CO[Si](C(C)C)(C(C)C)O[Si](C(C)C)(C(C)C)O[C@H]1CC(n1cnc3c(Cl)nc(NC(C)=O)nc31)S2)(CC)CC ; C#C[C@]1(CO)S[C@@H](n2cnc3c(OCC)nc(N)nc32)C[C@@H]1O]

A solution of sodium ethoxide (21.0%, 0.0608 mL, 0.163 mmol) and intermediate 2C (29.5 mg, 0.0407 mmol) and ethanol (1 mL) were stirred at room temperature for 90 minutes. The mixture was neutralized with acetic acid (1M, 1.6 mL, 0.163 mmol). The solvent was removed under reduced pressure and the residue was co-evaporated with toluene (5 mL). The residue was taken up in tetrahydrofuran (1 mL) and tetrabutylammonium fluoride (1 M, 0.305 mL, 0.305 mmol) was added. After 20 minutes acetic anhydride (0.20 mL, 0.200 mmol) was added. After 16 hours the reaction mixture was quenched with water (0.5 mL) and saturated sodium bicarbonate (3 mL). The mixture was extracted with ethyl acetate (3×10 mL). The organic phase was dried over sodium sulfate and the solvent was removed under reduced pressure. The residue was subjected to flash chromatography (0-30% methanol/dichloromethane). The fractions containing acylated products were combined and the solvent was removed under reduced pressure, providing a mixture of bis- and tris-acylated products (10 mg).

A mixture of the bis- and tris-acylated products from the previous reaction (18.2 mg), potassium carbonate (109 mg, 0.789 mmol) and methanol (4 mL) were stirred at 60° C. for 1 hour. The reaction mixture was cooled and neutralized with acetic acid (0.79 mL, 0.79 mmol). The solution was subjected to preparative HPLC (0-40% acetonitrile/water neutral). The fractions containing product were combined and subjected to lyophilization, providing compound4. ¹H NMR (400 MHz, DMSO-d₆) δ 8.29 (s, 1H), 7.02-6.14 (m, 2H), 6.11 (t, J=5.8 Hz, 1H), 5.91-4.81 (m, 2H), 4.46 (m, 3H), 3.87-3.63 (m, 2H), 3.39 (s, 1H), 2.63 (dt, J=13.1, 4.8 Hz, 1H), 2.59-2.52 (m, 1H), 1.36 (t, J=7.1 Hz, 3H). LCMS: MS m/z=335.811 [M+1].

### Example 5: ((2R,3S,5R)-5-(2-Amino-6-ethoxy-7H-purin-7-yl)-2-ethynyl-2-(hydroxymethyl)tetrahydrothiophen-3-ol) (5)

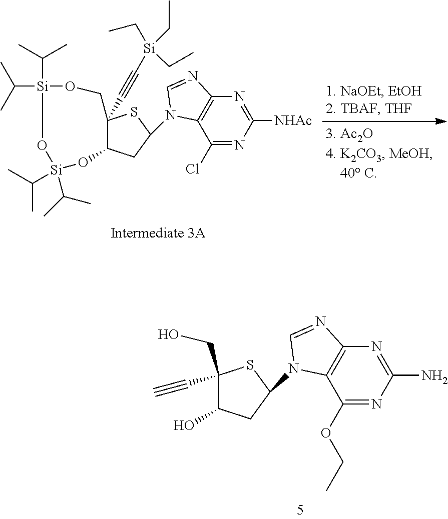 [SMILES: CC[Si](C#C[C@@]12CO[Si](C(C)C)(C(C)C)O[Si](C(C)C)(C(C)C)O[C@H]1CC(n1cnc3nc(NC(C)=O)nc(Cl)c31)S2)(CC)CC ; C#C[C@]1(CO)S[C@@H](n2cnc3nc(N)nc(OCC)c32)C[C@@H]1O]

A mixture of sodium ethoxide (21.0%, 0.155 mL, 0.414 mmol) and intermediate 3A (75 mg, 0.104 mmol) and ethanol (1 mL) was stirred at room temperature for 50 minutes. The mixture was neutralized with acetic acid (1M, 0.51 mL, 0.51 mmol). The solvent was removed under reduced pressure and the residue was co-evaporated with toluene (5 mL). The residue was taken up in tetrahydrofuran (1 mL) and tetrabutylammonium fluoride (1 M, 0.518 mL, 0.518 mmol) was added. After 20 minutes acetic anhydride (0.053 mL, 0.518 mmol) was added. After 16 hours the reaction mixture was quenched with water (0.5 mL) and saturated sodium bicarbonate (3 mL). The mixture was extracted with ethyl acetate (3×10 mL). The organic phase was dried over sodium sulfate and the solvent was removed under reduced pressure. The residue was subjected to flash chromatography (0-30% methanol/dichloromethane). The fractions containing acylated product were combined and the solvent was removed under reduced pressure, providing a mixture of bis- and tris-acylated products.

A mixture of the bis- and tris-acylated products from the previous reaction (40.6 mg, 0.0880 mmol), potassium carbonate (243 mg, 1.76 mmol) and ethanol (5 mL) was stirred at 60° C. for 1 hour. The reaction mixture was cooled and neutralized with acetic acid (1.76 mL, 1.76 mmol). The solution was subjected to preparative HPLC (0-40% acetonitrile/water neutral). The fractions containing product were combined and subjected to lyophilization, providing compound 5. ¹H NMR (400 MHz, DMSO-d₆) δ 8.82 (s, 1H), 7.64 (s, 2H), 6.17 (dd, J=5.6, 3.2 Hz, 1H), 5.70 (s, 2H), 4.56 (m, 2H), 4.31 (dd, J=9.5, 5.8 Hz, 1H), 3.71 (m, 2H), 3.48 (s, 1H), 2.62 (tt, J=9.0, 5.9 Hz, 2H), 1.42 (t, J=7.1 Hz, 3H). LCMS: MS m/z=335.901 [M+1].

### Example 6: (2-Ethylbutyl ((S)-(((2R,3S,5R)-5-(2,6-diamino-9H-purin-9-yl)-2-ethynyl-3-hydroxytetrahydrothiophen-2-yl)methoxy)(phenoxy)phosphoryl)-L-alaninate) (6)

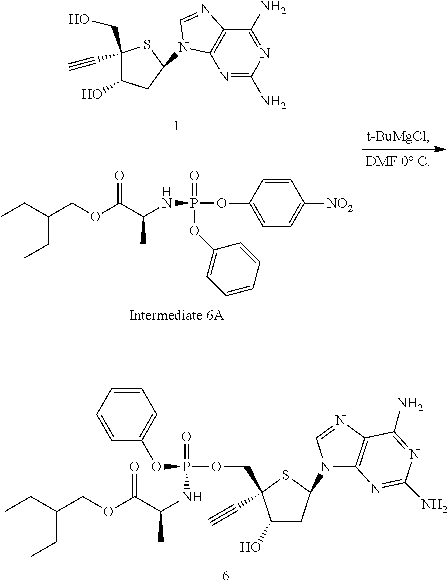 [SMILES: C#C[C@]1(CO)S[C@@H](n2cnc3c(N)nc(N)nc32)C[C@@H]1O ; CCC(CC)COC(=O)[C@H](C)N[P@](=O)(Oc1ccccc1)Oc1ccc([N+](=O)[O-])cc1 ; C#C[C@]1(CO[P@@](=O)(N[C@@H](C)C(=O)OCC(CC)CC)Oc2ccccc2)S[C@@H](n2cnc3c(N)nc(N)nc32)C[C@@H]1O]

tert-Butylmagnesium chloride (1 M, 31.3 μL, 0.0313 mmol) was added to a solution of 1 (4.8 mg, 0.0157 mmol) at 0° C. in N,N-dimethylformamide (1 mL). After 30 min 2-ethylbutyl (2S)-2-[[(4-nitrophenoxy)-phenoxy-phosphoryl]amino]propanoate (10.6 mg, 0.0235 mmol) was added. After 1 hour tert-Butylmagnesium chloride (1 M, 31.3 μL, 0.0313 mmol) and 2-ethylbutyl (2S)-2-[[(4-nitrophenoxy)-phenoxy-phosphoryl]amino]propanoate (10.6 mg, 0.0235 mmol) were added. After 5 h the reaction was neutralized with acetic acid (1 M, 62.7 μL, 0.0627 mmol). The mixture was subjected to preparative HPLC. The fractions containing product were combined and submitted to lyophilization. The resulting residue was subjected to flash chromatography (0-15% methanol/dichloromethane). The fractions containing product were combined and the solvent was removed under reduced pressure. The residue was taken up in acetonitrile/water and subjected to lyophilization, providing compound 6. ¹H NMR (400 MHz, Acetonitrile-d₃) δ 7.57 (s, 1H), 7.45-7.35 (m, 3H), 7.29 (dq, J=7.8, 1.2 Hz, 2H), 7.26-7.20 (m, 1H), 6.32 (t, J=7.7 Hz, 1H), 5.60 (s, 2H), 5.56 (dd, J=10.6, 5.7 Hz, 1H), 5.49 (s, 2H), 4.54 (q, J=4.0 Hz, 1H), 4.39-4.29 (m, 1H), 4.24 (dd, J=10.6, 4.0 Hz, 1H), 4.14-4.05 (m, 1H), 4.05-3.96 (m, 2H), 3.63 (d, J=4.7 Hz, 1H), 3.39 (ddd, J=12.7, 8.0, 4.0 Hz, 1H), 2.86 (s, 1H), 2.48 (ddd, J=13.9, 7.4, 3.4 Hz, 1H), 1.53-1.50 (m, 1H), 1.43-1.33 (m, 5H), 1.31 (d, J=10.1 Hz, 1H), 0.89 (td, J=7.5, 2.2 Hz, 6H). ³¹P NMR (162 MHz, Acetonitrile-d₃) δ 3.00. LCMS: MS m/z=617.996 [M+1].

### Example 7: (4-Amino-1-((2R,4S,5R)-5-ethynyl-4-hydroxy-5-(hydroxymethyl)tetrahydrothiophen-2-yl)-5-fluoropyrimidin-2(1H)-one) (7)

### Synthesis of Intermediate 7A (N-(5-fluoro-1-((6aR,9aR)-9-iodo-2,2,4,4-tetraisopropyl-6a-((triethylsilyl)ethynyl)tetrahydro-6H-thieno[3,2-f][1,3,5,2,4]trioxadisilocin-8-yl)-2-oxo-1,2-dihydropyrimidin-4-yl)acetamide)

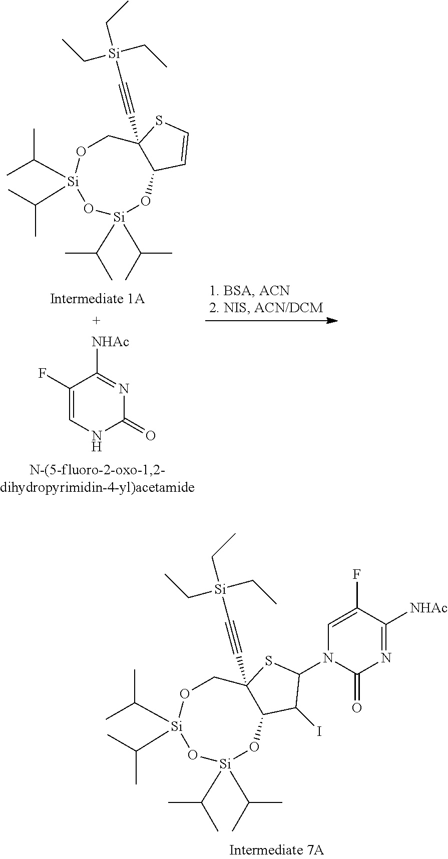 [SMILES: CC[Si](C#C[C@@]12CO[Si](C(C)C)(C(C)C)O[Si](C(C)C)(C(C)C)O[C@H]1C=CS2)(CC)CC ; CC(=O)Nc1nc(=O)[nH]cc1F ; CC[Si](C#C[C@@]12CO[Si](C(C)C)(C(C)C)O[Si](C(C)C)(C(C)C)O[C@H]1C(I)C(n1cc(F)c(NC(C)=O)nc1=O)S2)(CC)CC]

N,O-Bis(trimethylsilyl)acetamide (81 mg, 0.48 mmol) was added to a suspension of N-(5-fluoro-2-oxo-1,2-dihydropyrimidin-4-yl)acetamide (34.4 mg, 0.195 mmol) in acetonitrile (4 mL) and stirred at room temperature for 2 h. The reaction mixture was cooled to 0° C. and a solution of intermediate 1A (100 mg, 0.195 mmol) and N-Iodosuccinimide (70.2 mg, 0.312 mmol) in acetonitrile (2 mL), DCM (2 mL) was added drop-wise under Ar. The reaction mixture was stirred at 0° C. for 24 h. The reaction mixture was allowed to warm to room temperature, diluted with CHCl₃ (50 mL) and partitioned between a mixture of saturated aqueous sodium thiosulfate (25 mL) and saturated aqueous NaHCO₃ (25 ml). The aqueous phase was extracted with CHCl₃ (25 ml×2). The combined organic phases were dried over sodium sulfate and the solvent was removed under reduced pressure. The residue was subjected to flash chromatography (0-100% ethyl acetate/hexanes). The fractions containing product were combined and the solvent was removed under reduced pressure, to afford intermediate 7A. ¹H NMR (400 MHz, Acetonitrile-d₃) δ 8.54 (s, 1H), 8.42 (d, J=6.4 Hz, 1H), 6.24 (d, J=27.1 Hz, 1H), 4.82-4.70 (m, 1H), 4.06 (d, J=3.1 Hz, 2H), 3.69-3.64 (m, 1H), 2.38 (s, 3H), 1.20-0.94 (m, 37H), 0.71-0.58 (m, 6H). LCMS: MS m/z=809.63 [M+1].

### Synthesis of Intermediate 7B (N-(5-fluoro-2-oxo-1-((6aR,9aS)-2,2,4,4-tetraisopropyl-6a-((triethylsilyl)ethynyl)tetrahydro-6H-thieno[3,2-f][1,3,5,2,4]trioxadisilocin-8-yl)-1,2-dihydropyrimidin-4-yl)acetamide)

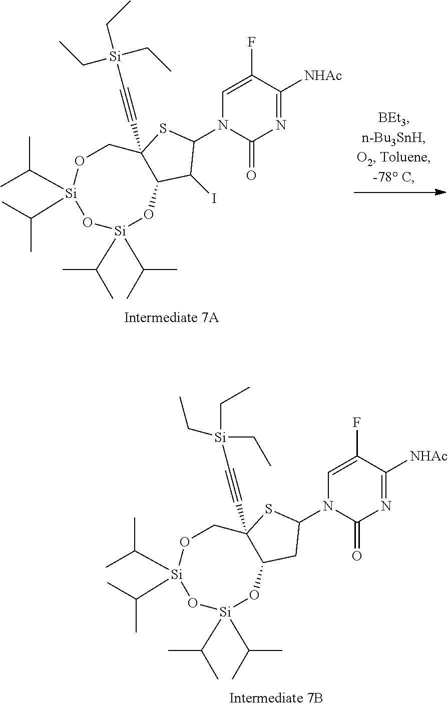 [SMILES: CC[Si](C#C[C@@]12CO[Si](C(C)C)(C(C)C)O[Si](C(C)C)(C(C)C)O[C@H]1C(I)C(n1cc(F)c(NC(C)=O)nc1=O)S2)(CC)CC ; CC[Si](C#C[C@@]12CO[Si](C(C)C)(C(C)C)O[Si](C(C)C)(C(C)C)O[C@H]1CC(n1cc(F)c(NC(C)=O)nc1=O)S2)(CC)CC]

To a solution of intermediate 7A (50 mg, 0.0.061 mmol) in toluene (4 mL) at −78° C. were added tributyltin hydride (28.7 mg, 0.098 mmol) followed by triethyl borane (0.012 mL, 0.0123 mmol, from 1M solution in Hexanes). This solution was flushed with oxygen and connected to an oxygen balloon and stirred at −78° C. for 8 h. The reaction mixture was quenched with a solution of methanol (2 mL) and water (0.5 mL) at −78° C. The mixture was warmed to room temperature, concentrated to dryness under reduced pressure and the product intermediate 7B was used without further purification in the next step. LCMS: MS m/z=684.32 [M+1].

### Synthesis of Intermediate 7C ((2R,3S)-5-(4-acetamido-5-fluoro-2-oxopyrimidin-1(2H)-yl)-2-(acetoxymethyl)-2-ethynyltetrahydrothiophen-3-yl acetate)

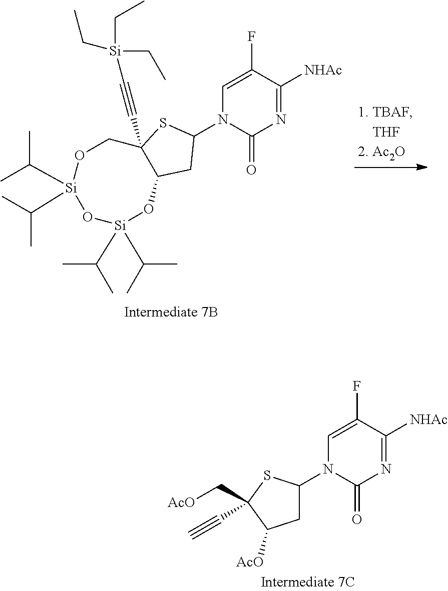 [SMILES: CC[Si](C#C[C@@]12CO[Si](C(C)C)(C(C)C)O[Si](C(C)C)(C(C)C)O[C@H]1CC(n1cc(F)c(NC(C)=O)nc1=O)S2)(CC)CC ; C#C[C@]1(COC(C)=O)SC(n2cc(F)c(NC(C)=O)nc2=O)C[C@@H]1OC(C)=O]

A solution of tetrabutylammonium fluoride (1M, 0.309 mL, 0.309 mmol) was added to a solution of intermediate 7B (42.2 mg, 0.062 mmol) in tetrahydrofuran (5 mL) and was stirred at room temperature for 3 h. After 3 h, acetic anhydride (25.2 mg, 0.247 mmol) was added and stirring was continued at room temperature overnight. The reaction mixture was partitioned between CHCl₃ and saturated aqueous NaHCO₃, the layers were separated, the aqueous layer was extracted with CHCl₃ (2×), the organic extracts were combined and dried over Na₂SO₄. The filtrate was concentrated to dryness under reduced pressure and used in the next step. ¹H NMR (400 MHz, Acetonitrile-d₃) δ 8.50 (s, 1H), 8.25 (d, J=6.6 Hz, 1H), 6.33-6.25 (m, 1H), 5.51-5.48 (m, 1H), 4.39 (s, 2H), 2.93 (s, 1H), 2.79-2.72 (m, 1H), 2.62-2.56 (m, 1H), 2.38 (s, 3H), 2.16 (s, 3H), 2.10 (s, 3H). LCMS: MS m/z=410.07 [M−1].

### Synthesis of (4-amino-1-((2R,4S,5R)-5-ethynyl-4-hydroxy-5-(hydroxymethyl)tetrahydrothiophen-2-yl)-5-fluoropyrimidin-2(1H)-one) (7)

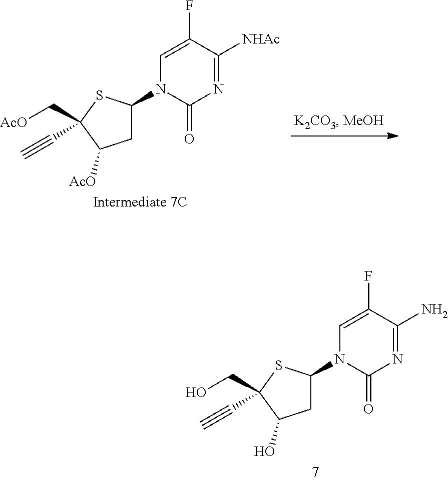 [SMILES: C#C[C@]1(COC(C)=O)S[C@@H](n2cc(F)c(NC(C)=O)nc2=O)C[C@@H]1OC(C)=O ; C#C[C@]1(CO)S[C@@H](n2cc(F)c(N)nc2=O)C[C@@H]1O]

To a solution of intermediate 7C (30 mg, 0.054 mmol), in MeOH (3 mL) at 0° C. was added potassium carbonate (15.1 mg, 0.109 mmol) in one portion and the mixture was allowed to warm to room temperature and stirred for 18 h. The reaction mixture was neutralized by drop-wise addition of a solution of AcOH/Water (1:1) to pH 7. The solution was filtered and subjected to prep HPLC. The product-containing fractions were combined and subjected to lyophilization, providing compound 7. ¹H NMR (400 MHz, Acetonitrile-d₃) δ 8.21 (d, J=7.2 Hz, 1H), 6.34-6.20 (m, 3H), 4.35 (dd, J=8.5, 4.7 Hz, 1H), 3.94-3.68 (m, 3H), 3.65 (d, J=24.9 Hz, 1H), 2.87 (s, 1H), 2.62-2.50 (m, 1H), 2.36-2.28 (m, 1H). ¹⁹F NMR (376 MHz, DMSO-d₆) δ −169.21. LCMS: MS m/z=285.79 [M+1].

### Example 8: (Hemitriethylamine ((2R,3S,5R)-5-(4-amino-5-fluoro-2-oxopyrimidin-1(2H)-yl)-2-ethynyl-3-hydroxytetrahydrothiophen-2-yl)methyl triphosphate) (8)

### Step 1:

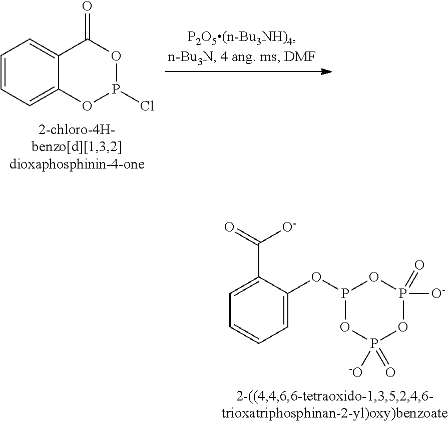

### Step 2:

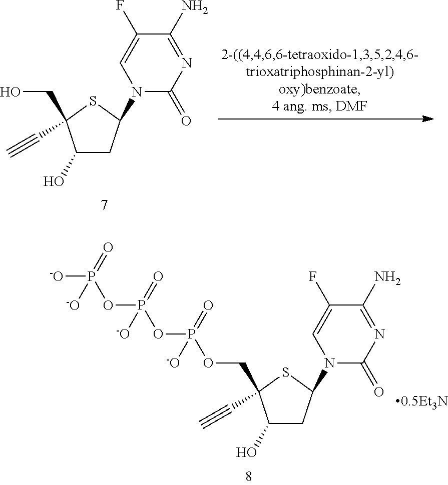

Step 1: An oven dried 40 mL vial was charged with a stir bar, activated 4 Å molecular sieves and connected to vacuum for 10 minutes and flushed with Argon. To this vial were added Tributylammonium pyrophosphate (86 mg, 0.48 mmol), DMF (0.75 mL) and dry tributylamine (0.618 mL, 2.59 mmol). The resulting mixture was transferred into another 40 mL vial containing activated 4 Å molecular sieves, dry 2-chloro-4H-benzo[d][1,3,2]dioxaphosphinin-4-one (27.6 mg, 0.136 mmol) and dimethylformamide (0.75 mL). The resulting mixture was stirred vigorously at 30° C. for 30 min to give a solution of 2-((4,4,6,6-tetraoxido-1,3,5,2,4,6-trioxatriphosphinan-2-yl)oxy)benzoate which was used directly in the next step.

Step 2: Compound7 (20 mg, 0.07 mmol) was placed into a round-bottom flask and dried under high vacuum overnight. To this flask was added activated 4 Å molecular sieves (25 mg) and the atmosphere was exchanged for argon. The solution of 2-((4,4,6,6-tetraoxido-1,3,5,2,4,6-trioxatriphosphinan-2-yl)oxy)benzoate from step 1 was transferred into the flask containing example 7 by syringe and the mixture was stirred at 25° C. for 4 h. The reaction progress was monitored by TLC (7:3 acetonitrile: 0.1 M ammonium chloride). Upon nearly complete consumption of the starting nucleoside, the mixture was cooled to 0° C., and quenched with drop-wise addition of an iodine solution [1 mL, of 3% in pyridine/water (9:1)]. After 15 minutes, triethylammonium bicarbonate buffer (1.0 M, 6 mL) was added at 0° C. and stirred for another 15 min. The volatiles were removed under reduced pressure (water bath temperature maintained <25° C.). The residue was re-dissolved in water (4 mL) and extracted with chloroform (6 mL). The collected aqueous layer was filtered and purified by preparative-HPLC to yield compound 8. LCMS: MS m/z=520.90 [M−4H]. ¹H NMR (400 MHz, Deuterium Oxide) δ 8.34 (d, J=6.5 Hz, 1H), 6.18 (t, J=5.7 Hz, 1H), 4.51 (dd, J=7.9, 4.6 Hz, 1H), 4.24-4.14 (m, 2H), 3.53-3.11 (m, 4H, from 0.6eq. triethylamine), 2.97 (s, 1H), 2.67-2.60 (m, 1H), 2.42-2.36 (m, 1H), 1.23 (dt, J=28.3, 7.3 Hz, 6H, from 0.6eq. triethylamine). ³¹P NMR (162 MHz, Deuterium Oxide) δ−10.80 (d, J=19.6 Hz), −12.15 (d, J=20.3 Hz), −23.12 (t, J=20.0 Hz).

### Example 9: 2-Ethylbutyl ((S)-(((2R,3S,5R)-5-(4-amino-5-fluoro-2-oxopyrimidin-1(2H)-yl)-2-ethynyl-3-hydroxytetrahydrothiophen-2-yl)methoxy)(phenoxy)phosphoryl)-L-alaninate (9)

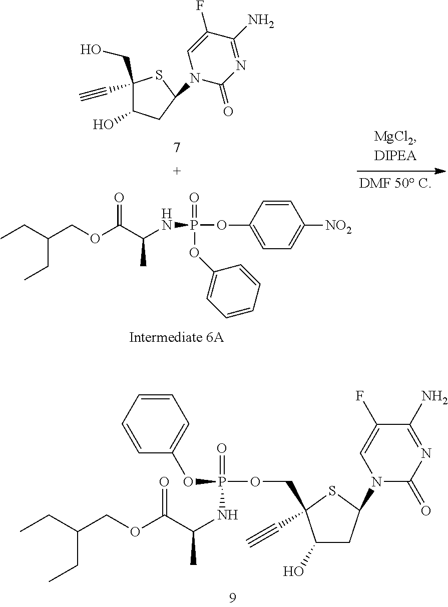 [SMILES: C#C[C@]1(CO)S[C@@H](n2cc(F)c(N)nc2=O)C[C@@H]1O ; CCC(CC)COC(=O)[C@H](C)N[P@](=O)(Oc1ccccc1)Oc1ccc([N+](=O)[O-])cc1 ; C#C[C@]1(CO[P@@](=O)(N[C@@H](C)C(=O)OCC(CC)CC)Oc2ccccc2)S[C@@H](n2cc(F)c(N)nc2=O)C[C@@H]1O]

Magnesium chloride (10.7 mg, 0.112 mmol) was added to a solution of 7 (8.0 mg, 0.028 mmol) in N,N-dimethylformamide (0.4 mL) followed by the addition of 2-ethylbutyl (2S)-2-[[(4-nitrophenoxy)-phenoxy-phosphoryl]amino]propanoate (25.3 mg, 0.056 mmol) and N,N-Diisopropylethylanine (0.02 mL, 0.112 mmol). After stirring for 1.5 hrs. at 50° C., additional magnesium chloride (1.5 mg, 0.014 mmol) and 2-ethylbutyl (2S)-2-[[(4-nitrophenoxy)-phenoxy-phosphoryl]amino]propanoate (6.3 mg, 0.014 mmol) were added. After stirring for another 2 hrs. at 50° C., the reaction mixture was cooled to room temperature, diluted with ethyl acetate and washed with 0.5M aqueous citric acid solution. The organic layer was separated, dried over Na₂SO₄ and filtered. The filtrate was concentrated to dryness under reduced pressure and the residue obtained was purified by preparative-HPLC to yield compound 9. ¹H NMR (400 MHz, Methanol-d4) δ 8.36 (d, J=6.7 Hz, 1H), 7.43-7.34 (m, 2H), 7.31-7.28 (m, 2H), 7.24-7.20 (m, 1H), 6.34-6.31 (m, 1H), 4.41-4.33 (m, 3H), 4.16-3.95 (m, 3H), 3.09 (s, 1H), 2.71-2.63 (m, 1H), 2.45-2.40 (m, 1H), 1.59-1.46 (m, 1H), 1.45-1.29 (m, 7H), 0.95-0.90 (m, 6H). LCMS: MS m/z=597.10 [M+1]. ³¹P NMR (162 MHz, Methanol-d4) δ 3.23.

### Example 9: Antiviral (HIV) Assay in MT-4 Cells

Compounds were tested in a high-throughput 384-well assay format for their ability to inhibit the replication of HIV-1 (IIIB) in MT-4 cells. Compounds were serially diluted (1:3) in DMSO on 384-well polypropylene plates and further diluted 200-fold into complete RPMI media (10% FBS, 1% P/S) using the Biotek Micro Flow and Labcyte ECHO acoustic dispenser. Each plate contained up to 8 test compounds, with negative (no drug) and positive (5 μM AZT) controls. MT-4 cells were pre-treated with 10 μL of either RPMI (mock-infected) or a fresh 1:250 dilution of HIV-1 IIIB concentrated virus stock. Infected and uninfected MT-4 cells were further diluted in complete RPMI media and added to each plate using a Micro Flow dispenser. After 5 days incubation in a humidified and temperature-controlled incubator (37° C.), Cell Titer Glo (Promega) was added to the assay plates and chemiluminescence read using an Envision plate-reader. EC₅₀ values were defined as the compound concentration that causes a 50% decrease in luminescence signal and were calculated using a sigmoidal dose-response model to generate curve fits and are shown in Table 1.

### Example 10: Cytotoxicity Assay in MT-4 Cells

Assays were performed as described in Example 9 except uninfected MT-4 cells were added to each well containing test compound. In addition, 10 μM puromycin was added to the last column of each assay plate to assess a base level of cytotoxicity. CC₅₀ values are shown in Table 1.

### Example 11: HIV RT Filter Binding IC₅₀ Assay

The RT IC₅₀ filter binding assay utilized a synthetic D19/D78mer annealed primer template complex prepared by incubating 50 μM of 19-mer [5′-G TCC CTG TTC GGG CGC CAC-3′] with 50 μM of 78-mer [3-CGA CCG TCC AGG GAC AAG CCC GCG GTG GCG ATC TCT TGA CAT TCC GTA ACC TTC GTA TTT TAA GCA TCA TAG TAC ACA-5] in 50 mM Tris pH 7.8 at 95° C. for 5 minutes, 55° C. for 15 minutes, and 37° C. for 10 minutes. 22.5 nM HIV RT was added to reaction mix containing 150 mM Tris-Cl pH 7.8, 180 mM KCl, 0.33 mM DTT, 0.9 mg/mL bSA, 12.6% glycerol, 3 uM D19/D78 mer. Serial dilutions of compounds were added at equal volumes and reactions were heated to 37° C. The reactions were initiated with equal volumes of dNTP mix containing 150 μM each of noncompeting dNTP and 1.2 μM of competing dNTP, and 0.1 μCi/μL competing dNTP. After 5 minutes at 37° C., 5 μL reactions were spotted on DE81 paper, washed three times in 0.125M Na₂HPO₄, dried, and quantified by autoradiography. IC₅₀ values were calculated in Prism by non-linear regression analysis using the dose-response (variable slope) equation (four-parameter logistic equation): Y=Bottom+(Top-Bottom)/(1+10\hat{ }((Log IC₅₀−X)*HillSlope)). RT IC₅₀ values are shown in Table 2.

### Example 12: DNA Polymerase γ Single Nucleotide Incorporation (SNI) Rate Assay

The DNA polymerase (pol) γ SNI assay utilized an annealed ³²P labeled primer-template complex of D19 [5′-G TCC CTG TTC GGG CGC CAC-3′] and D36 A/G/T/C [3′-CGA CCG TCC AGG GAC AAG CCC GCG (TGT/CGT/ACG/GCG) ATC TCT-5′] where the templating base was chosen to base pair to the natural dNTP and analogs assayed. 20 nM DNA pol γ large subunit and 100 nM pol γ accessory subunit were preincubated on ice for 5 minutes and added to reaction mix containing 50 mM Tris-Cl pH 8.0, 5 mM DTT, 0.2 mg/mL bSA, 200 nM D19/D36 mer and 10 mM MgCl₂. Reactions were heated to 37° C. and initiated by addition of 50 μM (final) natural dNTP or analogs, plus 50 μM dGTP or dCTP (for T and C or A and G templates respectively.) At 0, 5, 10, 30, 60, and 90 min, 10 μL of the reaction mixture was removed and quenched with 10 μL of a gel loading buffer containing 100 mM EDTA, 80% formamide and bromophenol blue, and heated at 65° C. for 5 minutes. The samples were run on a 20% polyacrylamide gel (8 M urea) and the gel was exposed to a phosphorimager screen. The substrate and the incorporation products, D19 and D20-28, were quantified using a Typhoon Trio Imager and Image Quant TL Software. The nucleotide incorporation rate was calculated in Prism by fitting the product formation using the single exponential equation: [D20]=A*(1−e−k*t), where [D20] represents the amount (in nM) of the elongated product formed, t represents the reaction time, k represents the observed rate, and A represents the amplitude of the exponential. Analog incorporation rates were reported as a percentage relative to the corresponding natural dNTP. DNA Pol γ SNIR values are shown in Table 2.

### Example 13: DNA Polymerase a Single Nucleotide Incorporation (SNI) Rate Assay

The DNA polymerase (pol) a SNI assay utilized an annealed ³²P labeled primer-template complex of D19 [5′-G TCC CTG TTC GGG CGC CAC-3′] and D36 A/G/T/C [3′-CGA CCG TCC AGG GAC AAG CCC GCG (TGT/CGT/ACG/GCG) ATC TCT-5′] where the templating base was chosen to base pair to the natural dNTP and analogs assayed. Reactions containing 50 mM Tris-Cl pH 8.0, 5 mM DTT, 0.2 mg/mL bSA, 200 nM D19/D36 mer and 10 mM MgCl₂ and 5 nM DNA pol a were heated to 37° C. and initiated by addition of 50 μM (final) natural dNTP or analogs. At 0, 5, 10, 20, 40, and 60 min, 10 μL of the reaction mixture was removed and quenched with 10 μL of a gel loading buffer containing 100 mM EDTA, 80% formamide and bromophenol blue, and heated at 65° C. for 5 minutes. The samples were run on a 20% polyacrylamide gel (8 M urea) and the gel was exposed to a phosphorimager screen. The substrate and the incorporation products, D19 and D20-28, were quantified using a Typhoon Trio Imager and Image Quant TL Software. The nucleotide incorporation rate was calculated in Prism by fitting the product formation using the single exponential equation: [D20]=A*(1−e−k*t), where [D20] represents the amount (in nM) of the elongated product formed, t represents the reaction time, k represents the observed rate, and A represents the amplitude of the exponential. Analog incorporation rates were reported as a percentage relative to the corresponding natural NTP. DNA Pol a SNIR values are shown in Table 2.

Compounds of the present disclosure demonstrate potent antiviral activity, as shown in Table 1 below. The compounds also demonstrate high selectivity over incorporation by host DNA polymerases, as shown in Table 2 below.

TABLE 1

| | MT4 HIV | MT4 |
|---|---|---|
| Compound | EC₅₀ (μM) | CC₅₀ (μM) |
| 1 | 0.683 | >25 |
| 2 | 4.52 | >25 |
| 3 | >25 | >25 |
| 4 | 0.477 | 23 |
| 5 | >5 | >25 |
| 6 | 0.026 | >25 |
| 7 | 0.018 | 24 |
| 9 | 0.145 | 23.17 |

TABLE 2

| | | DNA Pol γ | DNA Pol α |
|---|---|---|---|
| Compound | HIV RT IC50 | SNIR | SNIR |
| 8 | 0.21 | 1.3 | 2.6 |
| 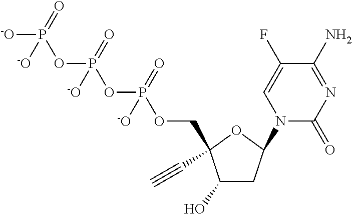 | 0.1 | 132.8 | 28.2 |

### Example 14: Rhesus Pharmacokinetic Studies

Male Indian Origin Rhesus Monkeys were used for the pharmacokinetic studies. Studies were conducted under a protocol approved by IACUC. Following overnight-fasting, monkeys were dosed intravenously with compound 7 at 1 mg/kg formulated as a solution in 15% (v/v) N-methylpyrrolidone, 35% (v/v) Polyethylene glycol 300, 50% (v/v) water. The intravenous dose was administered as an approximately 30-minute infusion, and after dose administration the dose apparatus was flushed with approximately 1 mL saline before removal from the animal. Food was returned 4 hours after dosing. Blood (0.5-mL) for plasma collection was drawn at pre-dose, 0.25, 0.48 (before end of infusion), 0.58, 0.75, 1, 1.5, 2, 4, 8, 12, 24, 48, 72, 144, and 168 hours post-dosing into K₂EDTA collection tubes. The K₂EDTA tubes were kept chilled at approximately 5° C. until centrifugation (4° C., 5 minutes, 2000×g) to collect plasma. Centrifugation began within 1 hour of collection. Plasma samples were harvested, placed in micronic tubes, and maintained on dry ice prior to storage at −70° C. until analysis by LC-MS/MS. Blood (6 mL) for peripheral blood mononuclear cells (PBMCs) collection was drawn at 12, 24, 48, 72, 144, and 168 hours post-dosing into sodium citrate collection tubes. The sodium citrate tubes were then gently inverted 3-4 times and stored upright at ambient temperature until centrifugation to collect PBMCs. Centrifugation began as soon as possible following collection, within 6 hours after collection. The PBMCs were separated by centrifugation (30 minutes at 1500×g-1800×g, 18-20° C.), washed once with ice cold 0.9% NaCl and treated with red blood cell lysis buffer (3.5 g ammonium chloride and 0.036 g ammonium carbonate in 500 mL ultrapure water, prepared before first blood collection) for 10 minutes at ambient temperature. Subsequently the cells were washed twice with ice cold 0.9% NaCl, the cell pellet resuspended in ice cold RIPA buffer (fortified with Pierce protease ad phosphatase inhibitor tablet), snap frozen in liquid nitrogen, and maintained at −70° C. until analysis by LC-MS/MS. Mean [±SD] plasma phaonacokinetic parameters for compound 7 after intravenous administration to fasted Indian Origin Rhesus monkeys at 1 mg/kg dose level ofcompound 7 are shown in Table 3. Mean [±SD] Intracellular Measured Concentrations and half-life of compound 7 and its triphosphate metabolite in peripheral blood mononuclear cells after intravenous administration to fasted Indian Origin Rhesus monkeys at 1 mg/kg dose level of compound 7 is shown in Table 4.

TABLE 3

| Compound | Dose | | | |
|---|---|---|---|---|
| Dosed & | Route, | AUC_{0-168 hr} | Cmax | t½ |
| Measured | level | (μM*hr) | (μM) | (hr) |
| 7 | Intravenous, | 14.63 ± 3.01 | 3.77 ± 0.31 | 50.02 ± 26.37 |
| | 1 mg/kg | | | |

TABLE 4

| | Compound 7 | Compound 8 |
|---|---|---|
| Time (hr) | Concentration (μM) | Concentration (μM) |
| 12 | BLQ | 2.19 ± 0.31 |
| 24 | BLQ | 1.34 ± 0.14 |
| 48 | BLQ | 0.42 ± 0.06 |
| 72 | BLQ | BLQ |
| 144 | BLQ | BLQ |
| 168 | BLQ | BLQ |

BLQ: below the limit of quantitation
Limit of quantitation for Compound 7 was 2.5 pmol/sample (equivalent to 2.5 μM in a sample with 5 million cells)
Limit of quantitation for triphosphate metabolite was 0.63 pmol/sample (equivalent to 0.63 μM in a sample with 5 million cells)
Calculated half life of triphosphate metabolite: 19.8 ± 9.3 hours

Following overnight-fasting, monkeys were administered oral compound 7 at 7.5 mg/kg formulated as a solution in 5% (v/v) N-methylpyrrolidone, 20% (v/v) Polyethylene glycol 300, 2% vitamin E TPGS, 73% (v/v) water. The oral dose was administered via oral gavage, and after dose administration the dose apparatus was flushed with approximately 10 mL water before removal from the animal. Food was returned 4 hours after dosing. Blood (0.5-mL) for plasma collection was drawn at pre-dose, 0.25, 0.5, 1, 2, 4, 6, 8, 12, 24, 48, 72, 120, 144, and 168 hours post-dose into K₂EDTA collection tubes. The K₂EDTA tubes were kept chilled at approximately 5° C. until centrifugation (4° C., 5 minutes, 2000×g) to collect plasma. Centrifugation began within 1 hour of collection. Plasma samples were harvested, placed in micronic tubes, and maintained on dry ice prior to storage at −70° C. until analysis by LC-MS/MS. Blood (6 mL) for peripheral blood mononuclear cells (PBMCs) collection was drawn at 12, 24, 48, 72, 120, and 168 hours post-dosing into sodium citrate collection tubes. The sodium citrate tubes were then gently inverted 3-4 times and stored upright at ambient temperature until centrifugation to collect PBMCs. Centrifugation began as soon as possible following collection, within 6 hours after collection. The PBMCs were separated by centrifugation (30 minutes at 1500×g-1800×g, 18-20° C.), washed once with ice cold 0.9% NaCl and treated with red blood cell lysis buffer (3.5 g ammonium chloride and 0.036 g ammonium carbonate in 500 mL ultrapure water, prepared before first blood collection) for 10 minutes at ambient temperature. Subsequently, the cells were washed twice with ice cold 0.9% NaCl and the cell pellet maintained at −70° C. until analysis by LC-MS/MS. Mean [±SD] plasma pharmacokinetic parameters for compound 7 after oral administration to fasted Indian Origin Rhesus monkeys at 7.5 mg/kg dose level of compound 7 are shown in Table 5. Mean [±SD] Intracellular Measured Concentrations and half-life of compound 7 and its triphosphate metabolite in peripheral blood mononuclear cells after oral administration to fasted Indian Origin Rhesus monkeys at 7.5 mg/kg dose level of compound 7 are shown in Table 6.

TABLE 5

| Compound | Dose | | | |
|---|---|---|---|---|
| Dosed & | Route, | AUC_{0-168 hr} | Cmax | t½ |
| Measured | level | (uM*hr) | (μM) | (hr) |
| compound | Oral, | 20.15 ± 14.88 | 1.5 ± 0.35 | 25.14 ± 16.31 |
| 7 | 7.5 mg/kg | | | |

TABLE 6

| | Compound 7 | Compound 8 |
|---|---|---|
| Time (hr) | Concentration (μM) | Concentration (μM) |
| 12 | 0.56 ± 0.03 | 1.43 ± 0.45 |
| 24 | 0.37 ± 0.20 | 1.78 ± 0.57 |
| 48 | BLQ | 1.15 ± 0.76 |
| 72 | BLQ | 0.47 ± 0.22 |
| 120 | BLQ | 0.17 ± 0.01 |
| 168 | BLQ | 0.09 ± 0.02 |

BLQ: below the limit of quantitation
Limit of quantitation for compound 7was 0.63 pmol/sample (equivalent to 0.16 μM in a sample with 20 million cells)
Limit of quantitation for triphosphate metabolite was 0.16 pmol/sample (equivalent to 0.04 μM in a sample with 20 million cells)
Calculated half-life of triphosphate metabolite: 43.1 ± 19.3 hours

1. A compound of Formula (I), 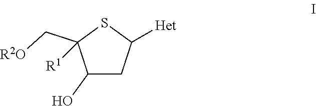
or a pharmaceutically acceptable salt thereof, wherein
R¹ is C₂-C₈ alkynyl;
R² is selected from H, 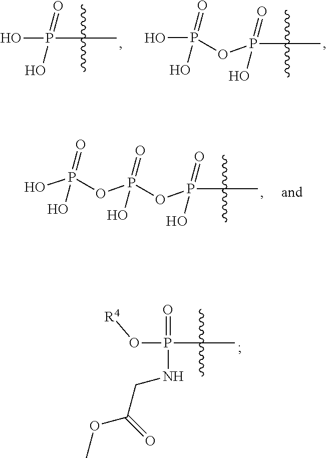
wherein:
R³ is C₁-C₈ alkyl;
R⁴ is selected from phenyl and naphthyl;
R⁵ is H or C₁-C₆ alkyl; and
Het is 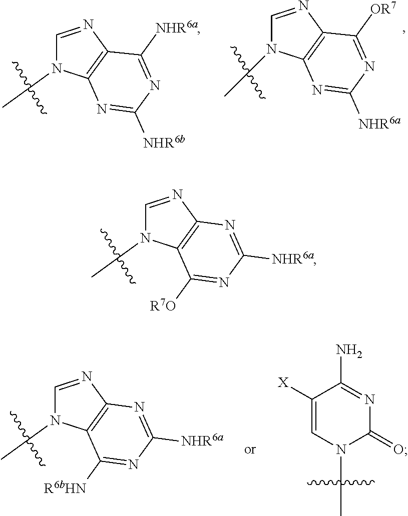
wherein:
X is halo;
R^{6a} is selected from H, C₁-C₆ alkyl, C₃-C₈ cycloalkyl, and C₁-C₆-alkylene-C₃-C₈ cycloalkyl;
R^{6b} is selected from H, C₁-C₆ alkyl, C₃-C₈ cycloalkyl, and C₁-C₆-alkylene-C₃-C₈ cycloalkyl; and
R⁷ is C₁-C₆ alkyl.

2. The compound of claim 1, or a pharmaceutically acceptable salt thereof, wherein
R¹ is

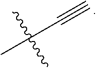 [SMILES: *C#C]

3. The compound of claim 1, or a pharmaceutically acceptable salt thereof, wherein
R² is H.

4. The compound of claim 1, or a pharmaceutically acceptable salt thereof, wherein
R² is selected from

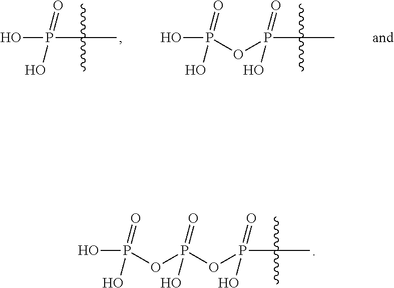 [SMILES: *P(=O)(O)O ; *P(=O)(O)OP(=O)(O)O ; *P(=O)(O)OP(=O)(O)OP(=O)(O)O]

5. The compound of claim 1, or a pharmaceutically acceptable salt thereof, wherein
R² is H or

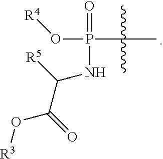

6. The compound of claim 1, or a pharmaceutically acceptable salt thereof, wherein
R² is

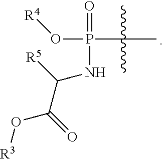

7. The compound of claim 1, or a pharmaceutically acceptable salt thereof, wherein
R² is

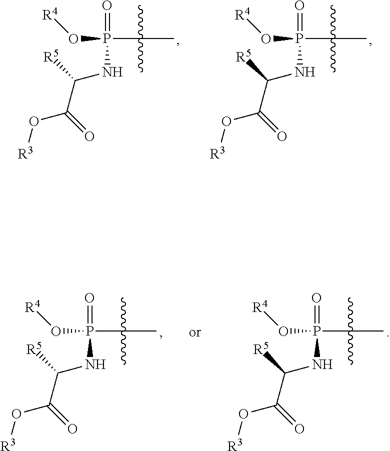

8. The compound of claim 1, or a pharmaceutically acceptable salt thereof, having the structure of Formula (II):

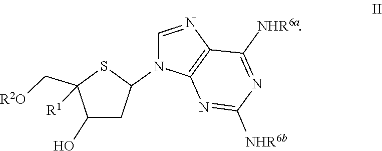

9. The compound claim 1, or a pharmaceutically acceptable salt thereof, having the structure of Formula (III):

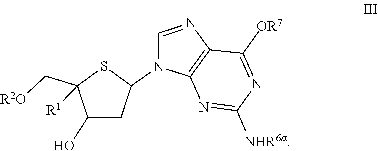

10. The compound of claim 1, or a pharmaceutically acceptable salt thereof, having the structure of Formula (IV):

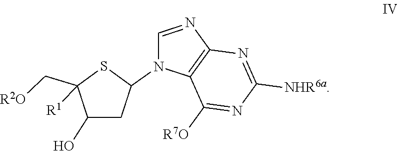

11. The compound of claim 1, or a pharmaceutically acceptable salt thereof, having the structure of Formula (V):

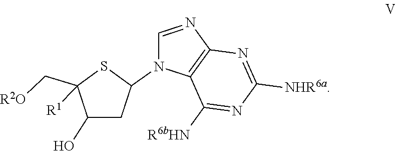

12. The compound of claim 1, or a pharmaceutically acceptable salt thereof, having the structure of Formula (VI):

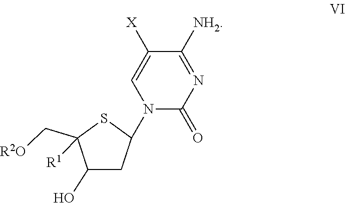

13. The compound of claim 12, or a pharmaceutically acceptable salt thereof, wherein X is F.

14. The compound of claim 1, or a pharmaceutically acceptable salt thereof, having the structure of Formula (VII):

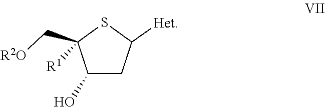

15. The compound of claim 14, or a pharmaceutically acceptable salt thereof, having the structure of Formula (VIIa) or (VIIb):

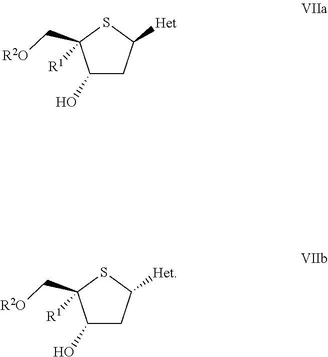

16. The compound of claim 14, or a pharmaceutically acceptable salt thereof, having the structure of Formula (VIII):

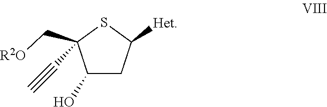

17. The compound of claim 14, or a pharmaceutically acceptable salt thereof, having the structure of Formula (IX):

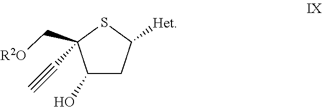

18. A compound, or a pharmaceutically acceptable salt thereof, with the structure shown below:

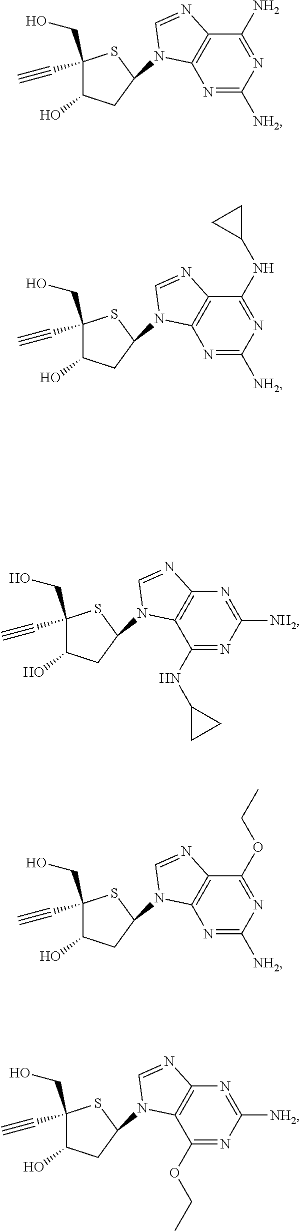

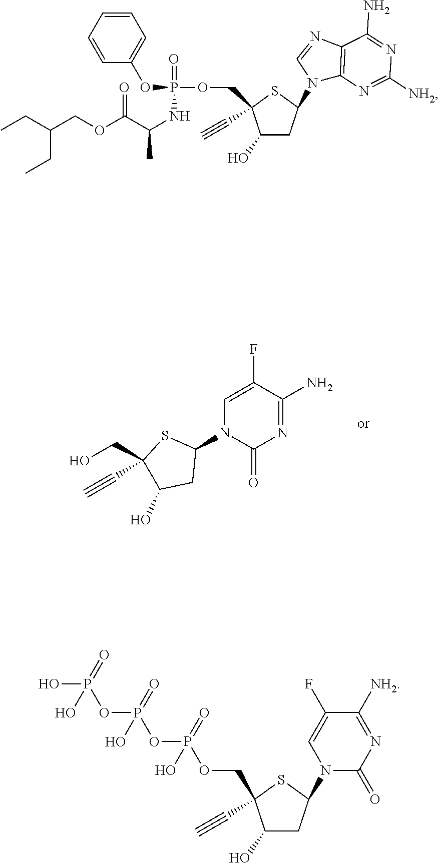 [SMILES: C#C[C@]1(CO[P@@](=O)(N[C@@H](C)C(=O)OCC(CC)CC)Oc2ccccc2)S[C@@H](n2cnc3c(N)nc(N)nc32)C[C@@H]1O ; C#C[C@]1(CO)S[C@@H](n2cc(F)c(N)nc2=O)C[C@@H]1O ; C#C[C@]1(COP(=O)(O)OP(=O)(O)OP(=O)(O)O)S[C@@H](n2cc(F)c(N)nc2=O)C[C@@H]1O]

19. A compound, or a pharmaceutically acceptable salt thereof, with the structure shown below:

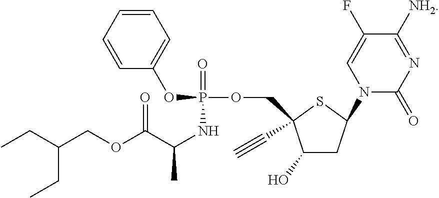 [SMILES: C#C[C@]1(CO[P@@](=O)(N[C@@H](C)C(=O)OCC(CC)CC)Oc2ccccc2)S[C@@H](n2cc(F)c(N)nc2=O)C[C@@H]1O]

20. A pharmaceutical composition comprising a therapeutically effective amount of a compound of any one of claims 1-19, or a pharmaceutically acceptable salt thereof, and a pharmaceutically acceptable carrier or excipient.

21. The pharmaceutical composition of claim 20 further comprising one or more additional therapeutic agents.

22. A method of treating a HIV comprising administering to a subject in need thereof a pharmaceutically effective amount of a compound of any one of claims 1-19, or a pharmaceutically acceptable salt thereof, or a pharmaceutical composition of claim 20 or 21.

23. The method of claim 22, wherein the compound or pharmaceutically acceptable salt thereof is administered in combination with an additional therapeutic agent.

24. The pharmaceutical composition of claim 21 or the method of claim 23, wherein the additional therapeutic agent comprises an integrase inhibitor (e.g., bictegravir, dolutegravir, cabotegravir, GS-6212), an HIV capsid inhibitor (e.g., lenacapavir) and/or a pharmaceutically acceptable salt thereof.
